(19) United States
(12) Patent Application Publication
(10) Pub. No.: US 2025/0120393 A1
(43) Pub. Date: Apr. 17, 2025

(54) MAGNETICALLY IMMOBILIZED BIOCIDAL ENZYMES AND BIOCIDAL CHEMICALS

(71) Applicant: ZYMtronix Catalytic Systems, Inc., Durham, NC (US)

(72) Inventors: Stephane Cedric Corgie, Ithaca, NY (US); Zachariah Robert Hansen, Ithaca, NY (US)

(73) Assignee: ZYMtronix Catalytic Systems, Inc., Durham, NC (US)

(21) Appl. No.: 18/895,058

(22) Filed: Sep. 24, 2024

Related U.S. Application Data
(63) Continuation of application No. 16/325,169, filed on Feb. 12, 2019, now Pat. No. 12,127,557.
(60) Provisional application No. 62/374,836, filed on Aug. 13, 2016.
(60) Provisional application No. 62/511,331, filed on May 25, 2017.

(51) Int. Cl. A01N 25/08; A01N 37/34; A01N 43/90; A01N 47/26; A01N 47/44; A01N 59/20; A01N 63/10; A01N 63/50; A61L 15/18; A61L 15/20; A61L 15/38; A61L 15/42; A61L 15/44; A61L 15/46

(52) U.S. Cl. CPC A01N 25/08; A01N 37/34; A01N 43/90; A01N 47/26; A01N 47/44; A01N 59/20; A01N 63/10; A01N 63/50; A61L 15/18; A61L 15/20; A61L 15/38; A61L 15/42; A61L 15/44; A61L 15/46; A61L 2300/11; A61L 2300/404; A61L 2300/406; A61L 2300/602

(57) ABSTRACT

The present invention provides compositions and methods for reducing microbial and nematodal contamination or infection in plants, animals, fabrics, and products therefrom. The present invention also provides compositions and methods for reducing human infections and the emergence of antimicrobial resistance. In particular, the invention provides magnetic nanoparticles comprising biocidal or biostatic enzymes in one component, substrates for the enzymes in a second component, and a biocidal chemical agent that works in combination or synergistically with the enzymes. The compositions are dormant and become active upon exposure to hydration, oxygen, or mixing.

### CROSS REFERENCE TO RELATED APPLICATIONS

This application is a continuation of U.S. application Ser. No. 16/325,169, filed Feb. 12, 2019, which is the National Phase Application of PCT/US17/45655 filed Aug. 6, 2017 and claims the benefit of U.S. Provisional Application No. 62/374,836, filed on Aug. 13, 2016 and 62/511,331, filed on May 25, 2017, each of which are incorporated herein by reference in their entirety.

### FIELD OF THE INVENTION

The present invention provides compositions and methods for reducing microbial and nematodal contamination or infection in plants, animals, fabrics, and products therefrom. The present invention also provides compositions and methods for reducing human infections and the emergence of antimicrobial resistance. In particular, the invention provides magnetic nanoparticles comprising biocidal or biostatic enzymes in one component, substrates for the enzymes in a second component, and a biocidal chemical agent that works in combination or synergistically with the enzymes. The compositions are dormant and become active upon exposure to hydration, oxygen, or mixing.

### BACKGROUND OF THE INVENTION

Contaminating and infectious microorganisms significantly reduce the yield, quality, and safety of agricultural and animal products worldwide. The resulting economic losses are in the hundreds of billions of dollars annually in the United States alone. In addition, current methods for reducing plant and animal infections rely heavily on the use of antimicrobial chemicals that may result in fungicide and antibiotic resistance that, in turn, increases the probability of selecting for drug-resistant plant, animal, and human pathogens. These microbes have been selected to survive in the presence of medically and agriculturally important antimicrobial chemicals and are a significant threat to human health and food security.

For example, Antibiotic Resistant Microbes (ARM) are a growing public health concern because infections have become increasingly difficult and expensive to treat. Concern turns into crisis in hospital environments. Antibiotics of last resort such as vancomycin, are steadily becoming ineffectual against superstrains. Carbapenem Resistant Enterobacteriaceae (CRE), some of the most ubiquitous microbes in the environment, are now resistant to almost all antibiotics. CRE infections are so difficult to treat that 50% of patients infected by them die. In the 2013 Antibiotic Resistance Threat report, the CDC identifies three major concerns: 1) new active molecules are harder to discover and produce, 2) development costs are prohibitive, and 3) resistance spreads faster than ever. The World Health Organization (WHO) warns that “[i]n the absence of urgent corrective and protective actions, the world is heading toward a post-antibiotic era, in which many common infections will no longer have a cure and [will], once again, kill unabated.” (World Health Day, Combat Drug Resistance: No Action Today Means No Cure Tomorrow, Statement by WHO Director-General, Dr. Margaret Chan, Apr. 6, 2011, http://www.who.int/mediacentre/news/statements/2011/whd_20110407/en/.) In 2013, the Center for Disease Control (CDC) estimated that 70 percent of the bacteria that caused hospital-acquired infections were resistant to at least one of the relevant antibiotics. (Antibiotic Resistance Threats in the United States, 2013, Centers for Disease Control and Prevention: Atlanta, GA, http://www.cdc.gov/drugresistance/threat-report-2013/.) It has long been argued by public advocacy groups, such as the Alliance for the Prudent Use of Antibiotics, that antibiotics are societal drugs. Individual use affects the entire community.

Such antibiotic resistance has become a worldwide concern now that the consequences of antibiotic overuse are being studied and reported. The CDC now estimates that in the U.S. at least 23,000 people die from multiple antibiotic-resistant bacteria infections every year. Id. In the U.S. alone, these superbug infections are responsible for $20 billion in excess healthcare costs, $35 billion in societal costs, and 8 million additional hospital stays each year. (Roberts et al., Clin. Infect. Dis. 49 (8): 1175-84 (2009).)

In agriculture, seeds can spread plant bacterial and fungal diseases across farms, states, and countries. In some instances, seedlings fail due to “damping off.” This is the death of a seedling before or shortly after emergence due to decomposition of the root and/or lower stem. Control of such diseases may begin with the seeds. Seed treatments should protect seeds from pathogens such as bacteria, viruses, and fungi. Thus, high-quality, disease-free seeds are an important part of obtaining higher plant yields and food safety.

In agriculture, bacteria can also contaminate animal environments in high density breeding operations. Pathogenic microbes such as Salmonella and Listeria entering the food chain cost hundreds of millions of dollars in product recalls and hospitalizations. (Hoffmann, S. and T. D. Anckwe, Making Sense of Recent Cost-of-Foodborne-Illness Estimates, United States Department of Agriculture, Economic Research Service, 2013, http://www.crs.usda.gov/publications/eib-economic-information-bulletin/eib118.aspx; Hoffmann et al., J. Food Prot. 75 (7): 1292-1302 (2012).)

Some food borne pathogens are also known to be become antibiotic-resistant. Foodborne pathogens have a vast impact on Americans, causing 48 million illnesses, 28,000 hospitalizations, and at least 3,000 deaths each year. (http://www.cdc.gov/foodborneburden/2011-foodborne-estimates.html.) They also have a tremendous impact on businesses and the healthcare system resulting in annual costs of $14-$16 billion. This includes direct medical costs and value of time lost to illness. Bacteria are the primary culprits. They comprise four of the top five pathogens that contribute to illness, three of the top five that cause hospitalizations, and three of the top five that cause mortality.

Nematodes are microscopic worms that cause eighty billion dollars of crop loss in the world each year. Plant-parasitic nematodes threaten crops throughout the world. In fact, all crops are damaged by at least one species of nematode. They attack almost every part of the plant including roots, stems, leaves, fruits and seeds.

Some 5,000 species of nematodes are estimated to be parasites of vertebrate animals and humans. These species are often characterized in a larger group of worm parasites as helminths. Strategies for managing nematode parasites of domestic vertebrate animals include control of secondary hosts or vectors and the use of chemical anthelminthics. Roundworms can infect dogs, cats, cattle, sheep, pigs, and poultry.

Most parasitic roundworms have direct life cycles, i.e. the free-living stages that do not need an intermediate host for development. They can directly infect their final host where they migrate to their predilection sites and complete development to adults. Inside the final host, pregnant females produce thousands of eggs that are usually excreted with the feces of the host and contaminate pastures, rivers, lakes, etc.

Controlling plant pathogens relies heavily on synthetic chemicals to maintain high product yields. The public has shown increasing concern, however, over the effects that agrochemical residues have on human health and the environment. (Mark et al., FEMS Microbiol. Ecol. 56 (2): 167-77 (2006); Ritter et al., J. Tox. Environ. Health 9 (6): 441-56 (2006).) Farmers who use synthetic agrochemicals have more neurological problems that include headaches, fatigue, insomnia, dizziness and hand tremors. (http://www.nichs.nih.gov/health/topics/agents/pesticides/). Agrochemicals may also cause birth defects, nerve damage, cancer, decreased sperm motility and acute poisoning (Moses, AAOHN J., 37 (3): 115-30 (1989); Reeves and Schafer Int'l J., Occup. Environ. Health 9 (1): 30-39 (2003); Carozza et al., Environ. Health Perspect. 116 (4): 559-65 (2008); U.S. Environmental Protection Agency, 2014, http://www.cpa.gov/pesticides/food/risks.htm). Furthermore, protecting crops from fungal pathogens is particularly challenging for organic crops on which synthetic antifungal chemicals cannot be used.

Fungicides and antibiotics are widely used in developed agricultural systems to control disease and safeguard crop yield and quality. Over time, however, resistance to many of the most effective fungicides and antibiotics has emerged and spread in pathogen populations (Lucas et al., Adv Appl Microbiol., 90:29-92 (2015)). The widespread practice of routinely dosing farm animals with antifungals and antibiotics is contributing to this threat. Much of this use is for preventing, rather than treating, disease. Drug-resistant microbes carried by farm animals can spread to humans through consumption of contaminated food, from direct contact with animals, or by environmental spread, for example, in contaminated water or soil. Antibiotic and fungicide resistant pathogens of humans and farm animals are emerging and spreading at a rate that may not be contained by the development of new drugs.

Thus there is a significant need for new methods of controlling fungal, bacterial, oomycete, and nematode pathogens that cause agricultural contamination or infections as well as human infections.

### SUMMARY OF THE INVENTION

The present invention provides compositions and methods for reducing microbial contamination or infection in plants, animals, fabrics, and products therefrom. The present invention also provides compositions and methods for reducing human infections. The present invention also reduces the emergence of resistance in microbes towards chemical biocides. In particular, the invention provides magnetic nanoparticles comprising microbiocidal and/or microbiostatic enzymes in one component, substrates for the enzymes in a second component, and a chemical microbiocidal agent that works synergistically with the enzymes. The compositions are dormant and become active upon exposure to hydration, oxygen, or mixing.

Thus, the invention provides a solid fungicidal composition, comprising; a first component having self-assembled mesoporous aggregates of magnetic nanoparticles comprising a hydrogen peroxide producing enzyme and a free radical producing enzyme; a second component having a first substrate for said hydrogen peroxide producing enzyme and a second substrate for said free radical producing enzyme; and a chemical fungicide; wherein said composition is essentially inactive, wherein exposure of said first and second components to hydration or oxygen activates said composition and results in said substrate for said hydrogen peroxide producing enzyme being oxidized into hydrogen peroxide, wherein said hydrogen peroxide acts as a substrate for said free radical producing enzyme, and wherein said free radicals are produced having fungicidal activities.

In some embodiments, said chemical fungicide is selected from the group consisting of mefenoxam, myclobutanil, chlorothalonil, prothioconazole, trifloxystrobin, propiconazole, mancozeb, and copper. In a preferred embodiment, said chemical fungicide is chlorothalonil. In another preferred embodiment, said chemical fungicide is mancozeb.

The invention provides a solid bactericidal composition, comprising; a first component having self-assembled mesoporous aggregates of magnetic nanoparticles comprising a hydrogen peroxide producing enzyme and a free radical producing enzyme; a second component having a first substrate for said hydrogen peroxide producing enzyme and a second substrate for said free radical producing enzyme; and a chemical antibiotic; wherein said composition is essentially inactive, wherein exposure of said first and second components to hydration or oxygen activates said composition and results in said substrate for said hydrogen peroxide producing enzyme being oxidized into hydrogen peroxide, wherein said hydrogen peroxide acts as a substrate for said free radical producing enzyme, and wherein said free radicals are produced having bactericidal activities.

In some embodiments, said chemical antibiotic is selected from the group consisting of ampicillin, streptomycin, vancomycin, and copper.

The invention provides a liquid fungicidal composition, comprising; a first component having self-assembled mesoporous aggregates of magnetic nanoparticles comprising a free radical producing enzyme; a second component having a substrate for said free radical producing enzyme and a hydrogen peroxide source; and a chemical fungicide; wherein said composition is essentially inactive, wherein mixing said first and second components activates said composition and results in said hydrogen peroxide source acting as a substrate for said free radical producing enzyme, and wherein said free radicals are produced having fungicidal activities.

In some embodiments, said chemical fungicide is selected from the group consisting of mefenoxam, myclobutanil, chlorothalonil, prothioconazole, trifloxystrobin, propiconazole, mancozeb, an essential oil, and copper. In a preferred embodiment, said chemical fungicide is chlorothalonil. In another preferred embodiment, said essential oil is tea tree oil (TTO).

The invention provides a liquid bactericidal composition, comprising; a first component having self-assembled mesoporous aggregates of magnetic nanoparticles comprising a free radical producing enzyme; a second component having a substrate for said free radical producing enzyme and a hydrogen peroxide source; and a chemical antibiotic; wherein said composition is essentially inactive, wherein mixing said first and second components activates said composition and results in said hydrogen peroxide source acting as a substrate for said free radical producing enzyme, and wherein said free radicals are produced having bactericidal activities.

In some embodiments, the final chemical fungicide concentration is between about 10% and 2500% of the half maximal effective concentration (EC₅₀). In other embodiments, the final chemical antibiotic concentration is between about 1 and 100% of the minimum inhibitory concentration (MIC) or minimum bactericidal concentration (MBC).

In other embodiments of the invention, the compositions and methods disclosed herein comprise microbiocidal compositions that comprise both a chemical antibiotic and a chemical fungicide.

In some embodiments of the invention, said mesoporous aggregates of magnetic nanoparticles have an iron oxide composition. In other embodiments of the invention, said mesoporous aggregates of magnetic nanoparticles have a magnetic nanoparticle size distribution in which at least 90% of magnetic nanoparticles have a size of at least about 3 nm and up to about 30 nm, and an aggregated particle size distribution in which at least about 90% of said mesoporous aggregates of magnetic nanoparticles have a size of at least about 10 nm and up to 500 nm. In other embodiments of the invention, said mesoporous aggregates of magnetic nanoparticles possess a saturated magnetization of at least 10 emu/g.

In some embodiments of the invention, said free-radical-producing enzyme and hydrogen peroxide producing enzyme are contained in said mesoporous aggregates of magnetic nanoparticles in up to about 100% of saturation capacity.

In some embodiments of the invention, said hydrogen peroxide generating enzyme is an oxidase. In other embodiments of the invention, said oxidase is glucose oxidase or alcohol oxidase.

The invention provides an agricultural product comprising the fungicidal and bactericidal compositions disclosed herein. In some embodiments, the invention provides a liquid pesticide product comprising the fungicidal and bactericidal compositions disclosed herein. In other embodiments, the invention provides a seed coating, comprising the fungicidal or bactericidal compositions disclosed herein. In some embodiments, the invention provides a seed comprising the seed coatings disclosed herein, wherein said seed is selected from the group consisting of vegetable, fruit, flower and field crop.

In preferred embodiments, said vegetable seed is selected from the group consisting of tomato, pea, onion, garlic, parsley, oregano, basil, cilantro, carrot, cabbage, corn, cucumber, radish, pepper, broccoli, cauliflower, cucumber, spinach, kale, chard, artichoke, and lettuce. In other preferred embodiments, said fruit seed is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia. In other preferred embodiments, said field crop is selected from the group consisting of corn, wheat, soybean, canola, sorghum, potato, sweet potato, yam, lentils, beans, cassava, coffee, hay, buckwheat, oat, barley, rape, switchgrass, elephant grass, beet, sugarcane, and rice. In other preferred embodiments, said flower seed is selected from the group consisting of annual, perennial, bulb, flowering woody stem, carnation, rose, tulip, poppy, snapdragon, lily, mum, iris, alstroemeria, pom, fuji, and bird of paradise.

The invention provides an animal bedding, comprising the fungicidal or bactericidal compositions disclosed herein.

The invention provides a wound dressing, comprising the fungicidal or bactericidal compositions disclosed herein.

The invention provides a fabric, comprising the fungicidal or bactericidal compositions disclosed herein.

The invention provides a method of improving a plant product yield, comprising exposing the improved seeds disclosed herein to hydration and oxygenation prior to or during the planting or germination of said plant.

The invention provides a method of improving an animal product yield, comprising exposing the improved animal bedding disclosed herein to hydration and oxygen prior to or during use by said animal. In a preferred embodiment, said hydration is from said animal's urine. In other preferred embodiments, said animal product is selected from the group consisting of live animals, milk, meat, fat, eggs, bodily fluids, blood, serum, antibodies, enzymes, rennet, bone, animal byproducts, and animal waste.

In other preferred embodiments, said animal is selected from the group consisting of cows, pigs, chickens, turkeys, horses, sheep, goats, donkeys, mules, ducks, geese, buffalo, camels, yaks, llama, alpacas, mice, rats, dogs, cats, hamsters, guinea pigs, reptiles, amphibians, parrots, parakeets, cockatiels, canaries, pigeons, doves, and insects.

The invention provides a method of reducing sepsis, comprising administering the improved wound dressings disclosed herein to a wound.

The invention provides a method of producing the fungicidal or bactericidal compositions disclosed herein, comprising formulating said first component with a matrix material selected from the group consisting of water-soluble cellulose derivatives, water-solvatable cellulose derivatives, alginate derivatives, and chitosan derivatives and formulating said second component with a matrix material selected from the group consisting of water-soluble cellulose derivatives, water-solvatable cellulose derivatives, alginate derivatives, and chitosan derivatives. In preferred embodiments, said first component is further subjected to spray drying, freeze drying, drum drying, pulse combustion drying, or rotary seed coating. In other preferred embodiments, said second component is further subjected to spray drying, freeze drying, drum drying, pulse combustion drying, or rotary seed coating.

The invention provides a method of reducing or eliminating fungal or bacterial growth, comprising spraying a substance with the liquid fungicidal or bactericidal compositions disclosed herein.

The invention provides a method of protecting an agricultural product from a pathogen, comprising exposing said product to the fungicidal or bactericidal compositions disclosed herein. In preferred embodiments, said pathogen is a plant, animal, or human pathogen. In other preferred embodiments, said pathogen is a fungus, oomycete, or bacterium. In more preferred embodiments, said fungus is selected from the group consisting of Rhizoctonia species and Fusarium species. In other preferred embodiments, said pathogen is a bacterium selected from the group consisting of Xanthomonas campestris, Clavibacter michiganensis, Acidovorax avenae, Pseudomonas viridiflava, Pseudomonas syringae, Escherichia coli, Salmonella species, and Listeria species. In other preferred embodiments, said pathogen is an oomycete selected from the group consisting of Pythium species and Phytophthora species

The invention provides a method of reducing or eliminating damping off in a plant, comprising exposing said plant to the fungicidal or bactericidal compositions disclosed herein.

The invention provides a solid nematocidal composition, comprising a first component having self-assembled mesoporous aggregates of magnetic nanoparticles comprising a hydrogen peroxide producing enzyme and a free radical producing enzyme and a second component having a first substrate for said hydrogen peroxide producing enzyme and a second substrate for said free radical producing enzyme; wherein said composition is essentially inactive, wherein exposure of said first and second components to hydration or oxygen activates said composition and results in said substrate for said hydrogen peroxide producing enzyme being oxidized into hydrogen peroxide, wherein said hydrogen peroxide acts as a substrate for said free radical producing enzyme, and wherein said free radicals are produced having fungicidal activities.

The invention provides a liquid nematocidal composition, comprising a first component having self-assembled mesoporous aggregates of magnetic nanoparticles comprising a free radical producing enzyme and a second component having a substrate for said free radical producing enzyme and a hydrogen peroxide source; wherein said composition is essentially inactive, wherein mixing said first and second components activates said composition and results in said hydrogen peroxide source acting as a substrate for said free radical producing enzyme, and wherein said free radicals are produced having fungicidal activities.

In a preferred embodiment, the nematocidal compositions further comprising an essential oil. In more preferred embodiments, said essential oil is selected from the group consisting of tea tree (TTO), aegle, ageratum, citrus, citronella, orange, pine, eucalyptus, marigold, geranium, lemongrass, orange, palmarosa, mint, peppermint, cinnamon, clove, rosemary, thyme, garlic, oregano, anise, cumin, turmeric, curcuma, caraway, fennel, onion, and patchouli oil. In a most preferred embodiment, said essential oil is TTO.

The invention provides an agricultural product, comprising the nematocidal compositions described herein. In other embodiments, the invention provides a liquid pesticide product comprising the nematocidal compositions described herein.

The invention provides a seed coating comprising the nematocidal compositions described herein. In some embodiments, said seed is selected from the group consisting of vegetable, fruit, flower and field crop. In preferred embodiments, said vegetable seed is selected from the group consisting of tomato, pea, onion, garlic, parsley, oregano, basil, cilantro, carrot, cabbage, corn, cucumber, radish, pepper, broccoli, cauliflower, cucumber, spinach, kale, chard, artichoke, and lettuce.

In other preferred embodiments, said fruit seed is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia.

In other preferred embodiments, said field crop is selected from the group consisting of corn, wheat, soybean, canola, sorghum, potato, sweet potato, yam, lentils, beans, cassava, coffee, hay, buckwheat, oat, barley, rape, switchgrass, elephant grass, beet, sugarcane, and rice.

In other preferred embodiments, said flower seed is selected from the group consisting of annual, perennial, bulb, flowering woody stem, carnation, rose, tulip, poppy, snapdragon, lily, mum, iris, alstroemeria, pom, fuji, and bird of paradise.

The invention provides an animal bedding, comprising the nematocidal composition described herein.

The invention provides a method of improving a plant product yield, comprising exposing the seed of described herein to hydration and oxygenation prior to or during the planting or germination of said plant.

The invention provides a method of improving an animal product yield, comprising exposing the animal bedding described herein to hydration and oxygen prior to or during use by said animal. In some embodiments, said hydration is from said animal's urine. In other embodiments, said animal product is selected from the group consisting of live animals, milk, meat, fat, eggs, bodily fluids, blood, serum, antibodies, enzymes, rennet, bone, animal byproducts, and animal waste. In other embodiments, said animal is selected from the group consisting of cows, pigs, chickens, turkeys, horses, sheep, goats, donkeys, mules, ducks, geese, buffalo, camels, yaks, llama, alpacas, mice, rats, dogs, cats, hamsters, guinea pigs, reptiles, amphibians, parrots, parakeets, cockatiels, canaries, pigeons, doves, and insects.

The invention provides a method of producing the nematocidal composition described herein, comprising formulating said first component with a matrix material selected from the group consisting of water-soluble cellulose derivatives, water-solvatable cellulose derivatives, alginate derivatives, and chitosan derivatives and formulating said second component with a matrix material selected from the group consisting of water-soluble cellulose derivatives, water-solvatable cellulose derivatives, alginate derivatives, and chitosan derivatives. In some embodiments, said first component is further subjected to spray drying, freeze drying, drum drying, pulse combustion drying, or rotary seed coating. In other embodiments, said second component is further subjected to spray drying, freeze drying, drum drying, pulse combustion drying, or rotary seed coating.

The invention provides a method of reducing or eliminating nematode growth, comprising spraying a substance with the liquid nematocidal compositions described herein.

The invention provides a method of protecting an agricultural product from a nematode, comprising exposing said product to the nematocidal compositions disclosed herein. In some embodiments, said nematode is selected from the group consisting of Meloidogyne species (spp.), Heterodera spp., Globodera spp., Pratylenchus spp., Helicotylenchus spp., Radopholus similis, Ditylenchus dipsaci, Rotylenchulus reniformis, Xiphinema spp, Aphelenchoides spp., Toxocara spp., Bursaphelenchus xylophilus, and Trichinella spiralis. In preferred embodiments, said nematode is a Meloidogyne spp., a Trichinella spiralis, or a Toxocara spp.

### BRIEF DESCRIPTION OF THE DRAWINGS

FIGS. 1A, 1B, and 1C. Effect of LP:GOx enzyme ratio and substrate concentration on dry enzyme disk bactericidal efficacy. The analysis was done twice and results were consistent across the two replicates. Data for a single analytical run is shown. The effect of the LP:GOx ratio on zones of inhibition (ZOI) of Xanthomonas campestris pv. vitians (Xcv) is shown at 1× (FIG. 1A) and 10× (FIG. 1B) substrate concentrations. Each bar represents the mean of two technical replicates. FIG. 1C shows the effect of 1× vs. 10× substrate concentrations averaged across all five LP:GOx enzyme ratios. Each bar represents the mean of ten replicates. Statistical analysis was done using Tukey's Honest Significant Difference (HSD), P<0.05).

FIG. 2. Effect of LP:GOx enzyme concentration on dry enzyme disk efficacy against fungal growth (Rhizoctonia solani and Fusarium graminearum). Each bar represents the mean of two replicates. Columns with an asterisk above them are significantly different (Tukey's HSD, P<0.05).

FIG. 3. Effect of three LP:GOx enzyme concentrations on growth of Pythium ultimum. Plate A is a control with dry substrate disk only. Plate B shows that the lowest enzyme concentration (0.0034× or 0.8 nM enzyme) did not inhibit growth of P. ultimum relative to the control. Plate C shows a medium enzyme concentration (0.68× or 161 nM enzyme) completely inhibited P. ultimum growth. Plate D shows a high enzyme concentration (2× or 476 nM enzyme) also completely inhibited P. ultimum growth.

FIG. 4. Reduction in P. ultimum growth relative to the untreated control following treatment by Daconil®+enzyme disk (black bars), Daconil® only (gray bars), and enzyme disk only (dotted line). Enzymes concentrations in enzyme disks were 81 nM.

FIG. 5. Reduction in P. aphanidermatum growth relative to the untreated control following treatment by Daconil®+enzyme disk (black bars), Daconil® only (gray bars), and enzyme disk only (dotted line). Enzymes concentrations in enzyme disks were 81 nM.

FIG. 6. Reduction in P. ultimum growth following treatment by Daconil®, enzyme disk, and Daconil®+enzyme disk. Enzyme concentrations in enzyme disks were 81 nM. Smaller colony diameters indicate greater oomycete growth inhibition. Plate A shows that a Daconil®-only treatment (0.0078% Daconil®) resulted in an 18% reduction in growth relative to the non-treated control. Plate B shows a non-treated control. Plate C shows that a 0.0078% Daconil®+enzyme disk treatment resulted in a 97% reduction in growth relative to the non-treated control, and a 32% synergistic effect compared to the additive effects of fungicide-only and enzyme disk-only treatments. Plate D shows that an enzyme disk-only treatment resulted in a 46% reduction in growth relative to the non-treated control.

FIG. 7. Reduction in P. aphanidermatum growth following treatment by Daconil®, enzyme disk, and Daconil®+enzyme disk. Enzymes concentrations in enzyme disks were 81 nM. Plate A shows that a Daconil®-only treatment (0.078% Daconil®) resulted in a 34% reduction in growth relative to the non-treated control. Plate B shows a non-treated control. Plate C shows that a 0.078% Daconil®+enzyme disk treatment resulted in a 100% reduction in growth relative to the non-treated control. The small white halo surrounding the culture plug is due to the application of Daconil® and is not mycelial growth. Plate D shows that an Enzyme disk-only treatment resulted in an 84% reduction in growth relative to the non-treated control.

FIG. 8. Enhanced activity of ampicillin with dry enzyme disk against Xcv. Enzymes concentrations in enzyme disks were 238 nM. Plate A shows that 100 μg ampicillin alone resulted in a zone of interference (ZOI) of 36 mm and no zone of clearance (ZOC). Sparse ampicillin-resistant colonies can be seen throughout the ZOI. Plate B shows that a 100 μg ampicillin plus dry enzyme disk resulted in a ZOC of 32.5 mm and an additional ZOI that extended 4.5 mm beyond the edge of the ZOC. Plate C shows that a dry enzyme disk alone resulted in a ZOC of 33 mm with no ZOI beyond the ZOC boundary. Plates A-C are visualized using reverse black and white imaging to enhance growth visualization.

FIG. 9: Percent of nematodes killed after three-day treatment incubation. Each bar represents the mean of three replicates.

FIGS. 10A and 10B: Effect of treatments on nematodes hatching from cysts (FIG. 10A) and eggs (FIG. 10B). Black bars show live nematode juveniles counted and white bars show dead nematode juveniles counted. Each bar represents the mean of three replicates.

### DETAILED DESCRIPTION OF THE INVENTION

The present invention provides compositions and methods for reducing microbial contamination or infection in plants, animals, fabrics, and products therefrom. This is accomplished, for the first time, by the synergy of chemical antimicrobial agents with multicomponent compositions comprising (1) a hydrogen peroxide producing (HPP) enzyme and a free radical producing (FRP) enzyme in self-assembled magnetic nanoparticles in one component and (2) substrates for the enzymes in another component. These magnetically-immobilized enzymes may be in solid or liquid compositions that are stable or inactive. Thus, they may be stored prior to or after incorporation into products. When the fungicidal activities are required, these multicomponent compositions are activated by exposure to hydration and/or oxygen. The HPP enzyme acts on substrates to produce hydrogen peroxide and, e.g. D-glucono-8-lactone. The FRP enzyme acts on the hydrogen peroxide and one or more further substrates to produce free radicals. The hydrogen peroxide and free radicals have antimicrobial properties. In alternative embodiments, hydrogen peroxide is provided as opposed to a hydrogen peroxide producing enzyme plus its substrates. The antimicrobial activities are activated by exposure to hydration and/or oxygen. The disclosures of Int'l Pub. Nos. WO2012122437 and WO2014055853 as well as Int'l Appl. No. PCT/US16/31419, incorporated by reference herein in their entirety.

Self-assembled mesoporous nanoclusters comprising entrapped peroxidases are highly active and robust. The technology is a powerful blend of biochemistry, nanotechnology, and bioengineering at three integrated levels of organization: Level 1 is the self-assembly of peroxidase and oxidase enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

MNP immobilization provides highly active and cost-effective peroxidases. Peroxidases are very potent enzymes yet notoriously difficult to deploy in industrial settings due to strong inhibition in presence of excess peroxide. NPs increase peroxidation activity and reduce their inhibition which renders them industrially useful. Additionally, the MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primar magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate, are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (FesO/O, hematite (α-Fe2θ 3), maghemite (γ-Fe2C>3), or a spinel ferrite according to the formula AB₂O₄, wherein A is a divalent metal (e.g., Xn²+, Ni²+, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a remanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values. The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m2/g.

MNPs, their structures, organizations, suitable enzymes, and uses are described in WO2012122437 and WO2014055853, incorporated by reference herein in their entirety.

The compositions and methods of the invention, among other things, reduce or eliminate plant death due to pathogens. In some embodiments, the invention reduces or eliminates “damping off.” The American Phytopathological Society defines damping-off as “the death of a seedling before or shortly after emergence due to decomposition of the root and/or lower stem; it is common to distinguish between pre-emergence damping-off and post-emergence damping-off. Pre-emergence damping-off occurs before a seedling emerges from the soil line. Post-emergence damping-off occurs shortly after a seedling emerges from the soil line. The disease is commonly caused by the fungus Rhizoconia solani and numerous species in the oomycete genus Pythium, although other fungi and oomycetes can contribute. The disease is not crop-specific and causes losses to all agricultural crops.

In other embodiments, the assemblies have antimicrobial properties against a wide array of pathogens. In some embodiments, the pathogens include pathogenic plant bacteria species such as Acidovorax avenae, Agrobacterium tumefaciens, Burkholderia andropogonis, Burkholderia caryophylli, Burkholderia glumae, Candidatus liberibacter, Candidatus Phytoplasma solani, Clavibacter michiganensis, Dickeya dadantii, Erwinia psidii, Pectobacterium atrosepticum, Pectobacterium betavasculorum, Pectobacterium carotovorum, Pectobacterium carotovorum subsp. betavasculorum, Pectobacterium wasabiae, Phytoplasma, Pseudomonas amygdali, Pseudomonas asplenii, Pseudomonas caricapapayae, Pseudomonas cichorii, Pseudomonas coronafaciens, Pseudomonas corrugate, Pseudomonas ficuserectae, Pseudomonas flavescens, Pseudomonas fuscovaginae, Pseudomonas helianthi, Pseudomonas marginalis, Pseudomonas oryzihabitans, Pseudomonas palleroniana, Pseudomonas papaveris, Pseudomonas salomonii, Pseudomonas savastanoi, Pseudomonas syringae, Pseudomonas tomato, Pseudomonas turbinellae, Pseudomonas viridiflava, Psyllid yellows, Ralstonia solanacearum, Rhodococcus fascians, Spiroplasma citri, Xanthomonas axonopodis, Xanthomonas campestris, Xanthomonas oryzae, Xylella fastidiosa, Escherichia coli, Salmonella enterica, Listeria monocytogenes, and other plant, animal, human, soilborne, and environmental pathogens.

In other embodiments, the assemblies have antimicrobial properties against non-plant pathogen bacteria including Escherichia coli, Brucella sp., Vibrio sp., Serrati asp., Nocardia sp., Leptospira sp., Mycobacterium sp., Clostridium sp., Bacillus sp., Pseudomonas sp. Staphylococcus sp., Neisseria sp., Haemophilus sp., Helicobacter sp., Mycoplasma sp., Pseudomonas sp. Treponema sp., and Yersinia sp.

In other embodiments, the fungicidal assemblies are effective against plant pathogenic fungi including genera such as Alternaria sp., Armillaria sp. Ascochyta sp., Aspergillus sp., Bipoloaris, Bjerkandera sp., Botrytis sp., Ceratobasidium sp., Cercospora sp., Chrysimyxa sp., Cladosporium sp., Cochliobolus sp., Coleosporium sp., Colletotrichum sp., Cylindrocladium sp., Cytospora sp., Diaporthe sp., Didymella sp., Drechslera sp., Erysiphe sp, Exobasidium sp., Fusarium sp., Ganoderma sp., Gibberella sp., Gymnospragium sp., Helicobasidium sp., Inonotus sp., Leptosphaeria sp., Leucostoma sp. Marasmius sp., Microspaera sp., Mucor sp., Mycosphaerella sp., Nectria sp., Oidium sp., Passalora sp., Pestalotiopsis sp., Phaeoramularia sp., Phoma sp., Phyllosticta sp., Pseudocercospora sp., Puccinia sp., Pyrenophora sp., Rhizoctonia sp., Rhizopus sp., Septoria sp., Sphaceloma sp., Stemphylium sp., Stigmina sp., Tilletia sp., Typhula sp., Uromyces sp., Ustilago sp., and Verticillium sp.

In other embodiments, the fungicidal assemblies are effective against plant pathogenic oomycetes including genera such as Aphanomyces sp., Bremia sp., Peronosclerospora sp., Peronospora sp., Phytophthora sp., Plasmopara sp., Pseudoperonospora sp., Pythium sp. and Sclerophthora sp. In preferred embodiments, the oomycetes are Phytophthora infestans, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, or Albugo candida.

A number of genera and species of nematodes are highly damaging to a great range of plants, including foliage plants, agronomic and vegetable crops, fruit and nut trees, turfgrass, and forest trees. Thus, in some embodiments, the assemblies of the invention are effective against nematodes such as Meloidogyne species (spp.), Heterodera spp., Globodera spp., Pratylenchus spp., Helicotylenchus spp., Radopholus similis, Ditylenchus dipsaci, Rotylenchulus reniformis, Xiphinema spp, Aphelenchoides spp., Toxocara spp., Bursaphelenchus xylophilus, and Trichinella spiralis.

In other embodiments, the invention is effective against plant viruses that include plant viruses such as Mosaic Viruses, Mottle Viruses, Begomoviruses, Carlaviruses, Carmoviruses, Criniviruses, Fabaviruses, Furoviruses, Machlomoviruses, Macluraviruses, Necroviruses, Potexviruses, Tenuiviruses, and Tospoviruses.

In some embodiments, the invention provides hydrogen peroxide producing (HPP) enzymes. In certain embodiments, the HPP enzymes are oxidases that may be of the EX 1.1.3 subgenus. In particular embodiments, the oxidase may be EC 1.1.3.3 (malate oxidase), EC 1.1.3.4 (glucose oxidase), EC 1.1.3.5 (hexose oxidase), EC 1.1.3.6 (cholesterol oxidase), EC 1.1.3.7 (aryl-alcohol oxidase), EC 1.1.3.8 (L-gulonolactone oxidase), EC 1.1.3.9 (galactose oxidase), EC 1.1.3.10 (pyranose oxidase), EC 1.1.3.11 (L-sorbose oxidase), EC 1.1.3.12 (pyridoxine 4-oxidase), EC 1.1.3.13 (alcohol oxidase), EC 1.1.3.14 (catechol oxidase), EC 1.1.3.15 (2-hydroxy acid oxidase), EC 1.1.3.16 (ecdysone oxidase), EC 1.1.3.17 (choline oxidase), EC 1.1.3.18 (secondary-alcohol oxidase), EC 1.1.3.19 (4-hydroxymandelate oxidase), EC 1.1.3.20 (long-chain alcohol oxidase), EC 1.1.3.21 (glycerol-3-phosphate oxidase), EC 1.1.3.22, EC 1.1.3.23 (thiamine oxidase), EC 1.1.3.24 (L-galactonolactone oxidase), EC 1.1.3.25, EC 1.1.3.26, EC 1.1.3.27 (hydroxyphytanate oxidase), EC 1.1.3.28 (nucleoside oxidase), EC 1.1.3.29 (Nacylhexosamine oxidase), EC 1.1.3.30 (polyvinyl alcohol oxidase), EC 1.1.3.31, EC 1.1.3.32, EC 1.1.3.33, EC 1.1.3.34, EC 1.1.3.35, EC 1.1.3.36, EC 1.1.3.37 D-arabinono-1,4-lactone oxidase), EC 1.1.3.38 (vanillyl alcohol oxidase), EC 1.1.3.39 (nucleoside oxidase, H₂O₂ forming), EC 1.1.3.40 (D-mannitol oxidase), or EC 1.1.3.41 (xylitol oxidase).

The invention provides Free Radical Producing (FRP) enzymes in one of the sequential components of the solid fungicidal compositions. In some embodiments, the FRP is a peroxidase. Peroxidases are widely found in biological systems and form a subset of oxidoreductases that reduce hydrogen peroxide (H₂O₂) to water in order to oxidize a large variety of aromatic compounds ranging from phenol to aromatic amines.

Peroxidases belong to the sub-genus EC 1.11.1. In certain embodiments, the EC 1.11.1 enzyme is The EC 1.11.1 enzyme can be more specifically, for example, EC 1.11.1.1 (NADH peroxidase), EC 1.11.1.2 (NADPH peroxidase), EC 1.11.1.3 (fatty acid peroxidase), EC 1.11.1.4, EC 1.11.1.5 (cytochrome-c peroxidase), EC 1.11.1.6 (catalase), EC 1.11.1.7 (peroxidase), EC 1.11.1.8 (iodide peroxidase), EC 1.11.1.9 (glutathione peroxidase), EC 1.11.1.10 (chloride peroxidase), EC 1.11.1.11 (L-ascorbate peroxidase), EC 1.11.1.12 (phospholipid-hydroperoxide glutathione peroxidase), EC 1.11.1.13 (manganese peroxidase), EC 1.11.1.14 (diarylpropane peroxidase), or EC 1.11.1.15 (peroxiredoxin).

In other embodiments, the peroxidase may also be further specified by function, e.g., a lignin peroxidase, manganese peroxidase, or versatile peroxidase. The peroxidase may also be specified as a fungal, microbial, animal, or plant peroxidase. The peroxidase may also be specified as a class I, class II, or class III peroxidase. The peroxidase may also be specified as a myeloperoxidase (MPO), cosinophil peroxidase (EPO), lactoperoxidase (LPO), thyroid peroxidase (TPO), prostaglandin H synthase (PGHS), glutathione peroxidase, haloperoxidase, catalase, cytochrome c peroxidase, horseradish peroxidase, peanut peroxidase, soybean peroxidase, turnip peroxidase, tobacco peroxidase, tomato peroxidase, barley peroxidase, or peroxidasin. In these particular embodiments, the peroxidase is a lactoperoxidase.

The lactoperoxidase/glucose oxidase (LP/GOX) antimicrobial system occurs naturally in bodily fluids such as milk, saliva, tears, and mucous (Bosch et al., J. Applied Microbiol., 89 (2), 215-24 (2000)). This system utilizes thiocyanate (SCN—) and iodide (I—), two naturally occurring compounds that are harmless to mammals and higher organisms (Welk et al. Archives of Oral Biology, 2587 (2011)). LP catalyzes the oxidation of thiocyanate and iodide ions into hypothiocyanite (OSCN—) and hypoiodite (OI—), respectively, in the presence of hydrogen peroxide (H₂O₂). The H₂O₂ in this system is provided by the activity of GOX on β-D-glucose in the presence of oxygen. These free radical compounds, in turn, oxidize sulfhydryl groups in the cell membranes of microbes (Purdy, Tenovuo et al. Infection and Immunity, 39 (3), 1187 (1983); Bosch et al., J. Applied Microbiol., 89 (2), 215-24 (2000), leading to impairment of membrane permeability (Wan, Wang et al. Biochemistry Journal, 362, 355-362 (2001)) and ultimately microbial cell death. Concentrations as low as 20 UM of hypothiocyanite and hypoiodite can result in inhibition of cell growth (Bosch, van Doorne et al. 2000). The LP/GOX system is effective on thiocyanate on its own; when paired with iodide, there is a synergistic effect that enhances biostatic and biocidal activity and extends the susceptible target range including Gram negative bacteria (e.g., E. coli, P. aerugenosa), Gram positive bacteria (e.g., S. aureus, Streptococcus spp.), and fungus (e.g., C. albicans) (Reiter, Marshall et al. Infection and Immunity, 13 (3), 800-807 (1976); Bosch et al., J. Applied Microbiol., 89 (2), 215-24 (2000); Welk et al. Archives of Oral Biology, 2587 (2011).) Furthermore, the LP/GOX system functions in two phases: (1) the generation and action of hypothiocyanite and hypoiodite on cell membranes, and then, when these compounds are depleted, (2) excess H₂O₂ builds up, enacting its own oxidative damage on cellular structures (Reiter, Marshall et al. 1976). The forgoing references are incorporated herein by reference in their entirety.

The enzyme system has been deployed and approved in the industry for biofilm control such as toothpaste and milk anti-spoiling agents. The system is largely non-specific and robust with few reaction requirements. One study found persistent biostatic and biocidal activity against Gram (−) and (+) bacteria and C. albicans after 18 months of re-inoculation every two months Bosch et al., J. Applied Microbiol., 89 (2), 215-24 (2000). The effective pH range is 3-7 with a peak LP activity at pH 5 (Reiter, Marshall et al. 1976; Purdy, Tenovuo et al. 1983). Higher activity is typically witnessed against bacteria at pH 3, but this is likely due to inhibition of growth by low pH (Reiter, Marshall et al. 1976). Other than pH, the only strict requirement for activity of the LP/GOX system is the presence of oxygen, without which GOX can't generate H₂O₂ from glucose. The forgoing references are incorporated herein by reference in their entirety.

LP/GOX has been described as a pesticide for microorganisms that include bacteria and fungi. (See U.S. Pat. No. 6,447,811, incorporated by reference herein in its entirety). Thus, in some embodiments, the invention described herein provides magnetically-immobilized pesticides in solid or liquid formulations. The pesticides comprise a peroxidase enzyme that produces a free radical. In some embodiments, the peroxidase enzyme is lactoperoxidase. The pesticides further comprise a peroxide source that may include an enzyme that oxidizes glucose.

In some embodiments of the invention, the chemical fungicide may be one or more of the following: mefenoxam, myclobutanil, chlorothalonil, prothioconazole, trifloxystrobin, propiconazole, mancozeb, Copper, methyl benzimidazole carbamates, dicarboximides, demethylation inhibitors (DMI), phenylamides (PA), amines, phosphorothiolates, dithiolanes, carboxamides, hydroxy-(2-amino-)pyrimidines, anilino-pyrimidines (AP), N-phenyl carbamates, quinone outside inhibitors (QOI), phenylpyrroles (PP), quinolines, aromatic hydrocarbons (AH), heteroaromatics, melanin biosynthesis inhibitors-dehydratase (MBI-D), hydroxyanilides, succinate biosynthesis inhibitors (SBI), polyoxins, phenylureas, quinone inside inhibitors (QiI), benzamides, enopyranuronic acid antibiotic, hexopyranosyl antibiotic, glucopyranosyl antibiotic, cyanoacetamide-oximes, carbamates, uncouplers of oxidative phosphorylation, organo tin compounds, carboxylic acids, heteroaromatics, phosphonates, phthalamic acids, benzotriazines, benzene-sulfonomides, pyridazinones, ATP production inhibitors, complex I of respiration inhibitors, carboxylix acid amides (CAA), tetracycline antibiotic, thiocarbamate, host plant defense inducers including salicylic acid pathway, fungicides with unknown target sites of action, fungicides with multi-site contact activity, mineral oils, organic oils, or potassium bicarbonate.

In some embodiments of the invention, a chemical antibiotic is used that may be one or more of the following: chemical families of aminoglycosides, ansamycins, carbacephems, carbapenems, cephalosporins, glycopeptides, lincosamides, lipopeptides, macrolides, monolactams, nitrofurans, oxazolidinones, penicillins, polypeptide antibiotics, quinolones, fluoroquinolones, sulfonamides, tetracyclines. aminoglycosides, ansamycins, carbapenems, cephalosporins, glycopeptides, lincosamides, lipopeptides, macrolides, monobactams, nitrofurans, oxazolidinones, penicillins, polypeptides, quinolones, rifamycins, streptogramins, sulfonamides, tetracyclines, tuberactinomycins, or drugs with activity against mycobacteria. In preferred embodiments, the chemical antibiotic is ampicillin.

The invention provides that the chemical fungicides and antibiotics (chemical microbiocides) may be measured by its minimum inhibitory concentration (MIC) in the compositions and methods described herein. The MIC is the lowest concentration of a chemical that prevents visible growth of a bacterium, fungus, or oomycete. The MIC of the microbiocides may be determined, for instance, by preparing solutions of the chemical at increasing concentrations, incubating the solutions with the separate batches of cultured bacteria, and measuring the results using agar dilution or broth microdilution

The minimum bactericidal concentration (MBC) is the lowest concentration of an antibacterial agent required to kill a particular bacterium. It can be determined from broth dilution minimum inhibitory concentration (MIC) tests by subculturing on agar plates that do not contain the test agent. The MBC is identified by determining the lowest concentration of antibacterial agent that reduces the viability of the initial bacterial inoculum by ≥99.9%. The MBC is complementary to the MIC; whereas the MIC test demonstrates the lowest level of antimicrobial agent that inhibits growth, the MBC demonstrates the lowest level of antimicrobial agent that results in microbial death. This means that even if a particular MIC shows inhibition, plating the bacteria onto agar might still result in organism proliferation because the antimicrobial did not cause death. Antibacterial agents are usually regarded as bactericidal if the MBC is no more than four times the MIC. Microorganisms may survive microbiocides because they develop resistance to them.

The final chemical fungicide or antibiotic in the invention is at a final concentration of less than 1% or about 1%, 2%, 3%, 4%, 5%, 6%, 7%, 8%, 9% 10%, 20%, 30%, 40%, 50%, 60%, 70%, 80%, 90%, 100% of the minimum inhibitory concentration (MIC) or minimum bactericidal concentration (MBC).

The chemical microbiocides in the invention can also be measured by their EC₅₀. The term half maximal effective concentration (EC₅₀) refers to the concentration of a drug, antibody or toxicant which induces a response halfway between the baseline and maximum after a specified exposure time. It is used herein as a measure of microbiocide potency. The EC₅₀ of a graded dose response curve therefore represents the concentration of a compound where 50% of its maximal effect is observed. The EC50 of a quantal dose response curve represents the concentration of a compound where 50% of the population exhibit a response after a specified exposure duration. The microbiocides in the invention, including the fungicides and anbiotics, is at a final concentration of less than 1% or about 1%, 2%, 3%, 4%, 5%, 6%, 7%, 8%, 9% 10%, 20%, 30%, 40%, 50%, 60%, 70%, 80%, 90%, 100%. In other embodiments, the final chemical microbiocidal concentration is between about 100% and 500%, 500% and 1000%, 1000% and 2000%, 2000% and 2500%, 2500% and 5000%, 5000% and 10,000%.

The invention provides inactive magnetically-immobilized enzymes. The enzymes may be inactive because they are not exposed to water, oxygen, substrates, or any combination thereof. In a preferred embodiment of the present invention, the magnetically-immobilized enzymes are in an oil base. This limits enzymatic activity prior to use. Activation of the immobilized enzymes occurs upon exposure to hydration and/or oxygen. In a more preferred embodiment, the magnetically-immobilized enzymes are in an oil base comprising an agent for emulsifying the oil in an aqueous solution to form an oil-in-water emulsion. In another more preferred embodiment, the oil is a mineral oil, vegetable oil, or animal oil. Exemplary mineral oils include paraffin oil and kerosene-type oils. Exemplary animal oils include fish oils such as herring and mackerel oil. Examples of vegetable oils are peanut oil, sesame oil, rape-seed oil, linseed oil, castor oil, soybean oil, corn germ oil, and cotton-seed oil.

In other embodiments, in order to further facilitate the distribution of the magnetically-immobilized enzymes over a surface, one or more spreading agents known in the art can further be added to the composition or the oil base. In some embodiments, the spreading agents are non-ionogenic surface tension-reducing substances. In preferred embodiments, the spreading agents are ethoxylated alcohols and phosphatidyl lipids.

In other embodiments, one or more adhesives can be added. Adhesives may help prevent the magnetically-immobilized enzymes from being rinsed off the plant by rain or other conditions. Adhesives are well known in the art. Examples are starch, gums such as xanthan gum, gum Arabic and carboxymethyl celluloses (CMCs).

The composition can be applied by means of coating, spraying, sprinkling, atomizing, overhead spraying, watering, immersing, and drip irrigation. A particularly advantageous method for applying the composition is spraying both by means of low volume methods (mist systems) and high volume methods. Drip irrigation can be used for culture systems on rockwool and other growth substrates. The magnetically-immobilized enzymes according to the invention can also be used to disinfect drip irrigation systems. In both latter cases the presence of the oil base is not strictly necessary for an optimal activity. Immersion in a bath with the composition is particularly suitable for the treatment of plant parts, in particular harvestable parts, such as bulbs, tubers, fruits and the like.

The magnetically-immobilized enzymes can be made commercially available in different forms. In a preferred embodiment, the peroxidase activity is delayed as long as possible because this increases the shelf-life of the product. The enzymatic activity starts upon exposure to both hydration (i.e. water) and oxygen. In the present case the glucose oxidase/glucose system is the hydrogen peroxide donor. In more preferred embodiments, the hydrogen peroxide donor is provided separately from the peroxidase. In addition, the oil base and the spreading agent can, if desired, also be packaged separately.

In another embodiment, a kit is provided for forming the composition the kit comprises an optionally concentrated enzyme composition comprising a peroxidase (e.g. lactoperoxidase) and a hydrogen peroxide donor (e.g. glucose oxidase and glucose). In preferred embodiments, the kit may further comprise thiocyanate, iodide, oil, an emulsifier, or spreading agents. In more preferred embodiments, the ingredients are mixed with each other before use. In another embodiment, the kit may comprise one or more ingredients in a concentrated form for dilution or hydration prior to or concurrently with use.

In embodiments where β-D-Glucose is oxidized to H₂O₂, or where cellulose derived sugars are oxidized to H₂O₂, cellulase enzymes may be provided with the compositions of the invention. In some embodiments, the seed coating further comprises the cellulase.

In some embodiments, the cellulases are exocellulases, endocellulases, hemicellulases, or combinations thereof known in the art. Endocellulase (EC 3.2.1.4) randomly cleaves internal bonds at amorphous sites that create new chain ends. Exocellulase (EC 3.2.1.91) cleaves two to four units from the ends of the exposed chains produced by endocellulase, resulting in the tetrasaccharides or disaccharides, such as cellobiose. There are two main types of exocellulases [or cellobiohydrolases (CBH)]—CBHI works processively from the reducing end, and CBHII works processively from the nonreducing end of cellulose. Cellobiase (EC 3.2.1.21) or beta-glucosidase hydrolyses the exocellulase product into individual monosaccharides. Oxidative cellulases depolymerize cellulose by radical reactions, as for instance cellobiose dehydrogenase (acceptor). Cellulose phosphorylases depolymerize cellulose using phosphates instead of water.

In other embodiments, endocellulases may include EC 3.2.1.4, endo-1,4-beta-D-glucanase, beta-1,4-glucanase, beta-1,4-endoglucan hydrolase, celluase A, cellulosin AP, endoglucanase D, alkali cellulase, cellulase A 3, celludextrinase, 9.5 cellulase, avicelase, pancellase SS, and 1,4-(1,3,1,4)-beta-D-glucan 4-glucanohydrolase). Cellulases enzymes are typically produced by fungi, bacteria, and protozoans of cellulose). Other names for ‘endoglucanases’ are: endo-1,4-beta-glucanase, carboxymethyl cellulase (CMCase), endo-1,4-beta-D-glucanase, beta-1,4-glucanase, beta-1,4-endoglucan hydrolase, and celludextrinase.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that it is expressed when properly transformed into a suitable host organism. Suitable host cells may be derived from bacteria, fungi, plants, or animals as is well-known in the art.

In some embodiments, the invention provides that the matrix material is a biopolymer. Examples include the polysaccharides (e.g., cellulose, hemicellulose, xylan, chitosan, inulin, dextran, agarose, and alginic acid), polylactic acid, and polyglycolic acid. In other embodiments, the matrix material is a water-soluble cellulose derivative, a water-solvatable cellulose derivative, an alginate derivative, and a chitosan derivative.

In some embodiments, the matrix comprises cellulose. Cellulose is an organic compound with the formula (C₆H₁₀O₅) n, a polysaccharide consisting of a linear chain of several hundred to many thousands of β(1→4) linked D-glucose units. The cellulose used in the invention may be obtained or derived from plant, algal, or microbial sources. In some embodiments, the invention provides cellulose derivatives known in the art. The hydroxyl groups (—OH) of cellulose can be partially or fully reacted with reagents known in the art. In preferred embodiments, the cellulose derivatives are cellulose esters and cellulose ethers (—OR). In more preferred embodiments, the cellulose derivatives are cellulose acetate, cellulose triacetate, cellulose proprionate, cellulose acetate proprionate (CAP), cellulose acetate butyrate (CAB), nitrocellulose (cellulose nitrate), cellulose sulfate, methylcellulose, ethylcellulose, ethyl methyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose, and carboxymethyl cellulose (CMC).

In some embodiments, the matrix comprises carboxymethyl cellulose. Carboxymethyl cellulose (CMC) or cellulose gum [1] is a cellulose derivative with carboxymethyl groups (—CH2-COOH) bound to some of the hydroxyl groups of the glucopyranose monomers that make up the cellulose backbone. It is synthesized using techniques known in the art, e.g., by the alkali-catalyzed reaction of cellulose with chloroacetic acid. The polar (organic acid) carboxyl groups render the cellulose soluble and chemically reactive. The functional properties of CMC depend on the degree of substitution of the cellulose structure (i.e., how many of the hydroxyl groups have taken part in the substitution reaction), as well as the chain length of the cellulose backbone structure and the degree of clustering of the carboxymethyl substituents.

In some embodiments, the matrix comprises hydroxypropyl cellulose (HPC). HPC is a derivative of cellulose with both water solubility and organic solubility. HPC is an ether of cellulose in which some of the hydroxyl groups in the repeating glucose units have been hydroxypropylated forming —OCH2CH(OH)CH3 groups using propylene oxide. The average number of substituted hydroxyl groups per glucose unit is referred to as the degree of substitution (DS). Complete substitution would provide a DS of 3. Because the hydroxypropyl group added contains a hydroxyl group, this can also be etherified during preparation of HPC. When this occurs, the number of moles of hydroxypropyl groups per glucose ring, moles of substitution (MS), can be higher than 3. Because cellulose is very crystalline, HPC must have an MS about 4 in order to reach a good solubility in water. HPC has a combination of hydrophobic and hydrophilic groups, so it has a lower critical solution temperature (LCST) at 45° C. At temperatures below the LCST, HPC is readily soluble in water; above the LCST, HPC is not soluble. HPC forms liquid crystals and many mesophases according to its concentration in water. Such mesophases include isotropic, anisotropic, nematic and cholesteric. The last one gives many colors such as violet, green and red.

In some embodiments, the matrix comprises methyl cellulose. Methyl cellulose (or methylcellulose) is derived from cellulose. It is a hydrophilic white powder in pure form and dissolves in cold (but not in hot) water, forming a clear viscous solution or gel. Methyl cellulose does not occur naturally and is synthetically produced by heating cellulose with caustic solution (e.g. a solution of sodium hydroxide) and treating it with methyl chloride. In the substitution reaction that follows, the hydroxyl residues (—OH functional groups) are replaced by methoxide (—OCH3 groups).

Different kinds of methyl cellulose can be prepared depending on the number of hydroxyl groups substituted. Cellulose is a polymer consisting of numerous linked glucose molecules, each of which exposes three hydroxyl groups. The Degree of Substitution (DS) of a given form of methyl cellulose is defined as the average number of substituted hydroxyl groups per glucose. The theoretical maximum is thus a DS of 3.0, however more typical values are 1.3-2.6.

In some embodiments, the matrix comprises alginate. Alginate, also called Alginic acid, and algin, is an anionic polysaccharide distributed widely in the cell walls of brown algae. When bound with water it forms a viscous gum. In extracted form it absorbs water quickly; it is capable of absorbing 200-300 times its own weight in water. It is sold in filamentous, granular or powdered forms. The invention provides matrix materials of known alginate and alginate-derived materials. In preferred embodiments, the alginate-derived materials include alginate-polylysine-alginate (APA), Alginate/Poly-l-lysine/Pectin/Poly-1-lysine/Alginate (APPPA), Alginate/Poly-1-lysine/Pectin/Poly-l-lysine/Pectin (APPPP), and Alginate/Poly-L-lysine/Chitosan/Poly-l-lysine/Alginate (APCPA), alginate-polymethylene-co-guanidine-alginate (A-PMCG-A), hydroxymethylacrylate-methyl methacrylate (HEMA-MMA), multilayered HEMA-MMA-MAA, polyacrylonitrile-vinylchloride (PAN-PVC).

In some embodiments, the matrix comprises chitosan. Chitosan is a linear polysaccharide composed of randomly distributed β-(1-4)-linked D-glucosamine (deacetylated unit) and N-acetyl-D-glucosamine (acetylated unit). The amino group in chitosan has a pKa value of ˜6.5, which leads to a protonation in acidic to neutral solution with a charge density dependent on pH and the % DA-value. This makes chitosan water soluble and a bioadhesive which readily binds to negatively charged surfaces such as mucosal membranes. It is produced commercially by deacetylating chitin, which is the structural element in the exoskeleton of crustaceans (such as crabs and shrimp) and cell walls of fungi, with sodium hydroxide. Chitosan is used in agriculture as a seed treatment and biopesticide. In winemaking, it is used as a fining agent, also helping to prevent spoilage. It is also used in bandages to reduce bleeding and as an antibacterial agent. It is also be used to help deliver drugs through the skin.

In other embodiments, the matrix materials may be acrylonitrile/sodium methallylsuflonate, (AN-69), polyethylene glycol/poly pentamethylcyclopentasiloxane/polydimethylsiloxane (PEG/PD5/PDMS), poly JVjiV-dimethyl acrylamide (PDMAAm), siliceous encapsulates, and cellulose sulphate/sodium alginate/polymethylene-co-guanidine (CS/A/PMCG).

In some embodiments, the invention provides antimicrobial compositions that are used, inter alia, for seed coatings. Any seeds that are vulnerable to pathogens that respond to the enzyme systems disclosed herein would benefit. In some embodiments, the seeds may be for vegetables, fruits, field crops, and flowers. In other embodiments, the invention provides antimicrobial compositions that are used, inter alia, for bedding for industrially or commercially relevant domesticated animals and products derived therefrom. Many domesticated animals are known in the art. In other embodiments, the invention provides fungicidal compositions that are used, inter alia, for wound dressings. Many wound dressings are known in the art. The invention provides fabrics that resist pathogens or contaminants that respond to the enzyme systems disclosed herein. The fabrics comprise the fungicidal compositions described herein.

Some embodiments of the invention provides compositions and methods for reducing human infections. This is accomplished, for the first time, by a multicomponent composition comprising a hydrogen peroxide producing (HPP) enzyme and a free radical producing (FRP) enzyme in magnetic nanoparticles in one component and substrates for the enzymes in another component. The solid compositions are stable and inactive. Thus, they may be stored prior to or after incorporation into products. When the fungicidal activities are required, the multicomponent compositions are activated by hydration. The HPP enzyme acts on substrates to produce hydrogen peroxide and D-glucono-8-lactone. The FRP enzyme acts on the hydrogen peroxide and one or more further substrates to produce free radicals. The hydrogen peroxide and free radicals have fungicidal properties.

In order that the invention described herein may be more fully understood, the following examples are set forth. It should be understood that these examples are for illustrative purposes only and are not to be construed as limiting this invention in any manner.

### Example 1—Microbiocidal Optimization of Dry Enzyme and Substrate Disks

### Materials and Methods

Five different lactoperoxidase (LP) to glucose oxidase (GOx) molar ratios were analyzed in dry enzyme disks to determine the optimal enzyme ratio for maximizing bactericidal activity. Two different concentrations of substrates in the dry substrate disks were also analyzed. These analyses were performed in-vitro on the bacterium Xanthomonas campestris pv. vitians isolate ‘09131A’ which was originally collected in New York State and isolated and identified by Christine Smart, Professor of Plant Pathology at Cornell University. (http://blogs.cornell.edu/smartlab/.)

A second analysis was done to determine the optimal enzyme concentration to inhibit fungal growth. Four concentrations of enzymes were analyzed in dry enzyme disks to determine the optimal enzyme concentration to maximize fungicidal activity. All analyses were performed in-vitro on plant-pathogenic microorganism cultures obtained from collaborators in the Cornell University Section of Plant Pathology and Plant-Microbe Biology (Cornell University, Ithaca, NY). The fungi Rhizoctonia solani (isolate ‘AC1-A1’) and Fusarium graminearum (isolate ‘GZ014NY98’) were collected in New York State isolated and identified by Professors Eric Nelson and Gary Bergstrom, respectively. The oomycete Pythium ultimum (isolate ‘Geneva16’) was collected in New York State and isolated and identified by Professor Eric Nelson. https://pppmb.cals.cornell.edu/people/eric-nelson.

A third analysis was done to determine the optimal enzyme concentration to inhibit oomycete growth. Three concentrations of enzymes were analyzed to determine optimal activity against oomycetes. The oomycete Pythium ultimum (isolate ‘Geneva16’) was collected in New York State and isolated and identified by Professor Eric Nelson (Cornell University, Ithaca, NY).

For the optimization of enzyme ratios for bactericidal activity, lactoperoxidase (LP) and glucose oxidase (GOx) enzymes were immobilized in five different LP:GOx molar ratios (see Table 1). Dry enzyme disk compositions were the same as those listed in Example 2 (Table 2) with the exception of enzyme concentrations, which are listed in Table 1. Having a 1:1 LP:GOx enzyme disk was considered as a standard, therefore GOx concentrations were decreased proportionally to create 10:1 and 5:1 disks and LP concentrations were decreased proportionally to create the 1:5 and 1:10 disks. Magnetic nanoparticles (pH 3) were mixed with LP+GOx enzyme mixtures (pH 7.4) in a 1:1 volume ratio to immobilize enzymes. Nanoparticle concentrations were adjusted to maintain a 30% enzyme:nanoparticle mass ratio (Table 1). Two dry substrate disk formulations that were analyzed, called 1× and 10×, are listed in Table 2. Enzyme disk and substrate disk components were mixed with distilled deionized H₂O to achieve desired concentrations, and dried under vacuum with a desiccant to remove H₂O. This process was repeated for the creation of all dry enzyme and substrate disks.

Magnetic nanoparticles were made and used for the molecular entrapment of peroxidase as described in US20150252352; PCT/US16/31419; and Corgié et al., Adv. Functional Materials 22:1940-51 (2012). The foregoing are incorporated by reference herein in their entirety.

Analysis to optimize bactericidal efficacy of dry enzyme and substrate disk formulations was performed twice. Each treatment was performed in duplicate. There were a total of ten treatments included in the LP:GOx ratio optimization. Each of the five LP:GOx ratios was analyzed with 1× and 10× substrate disks. Each analysis also included controls of 1× and 10× substrate disks only. The analyses were set up by creating bacterial lawns of Xcv on 85-mm petri dishes containing LB agar. 100 μl of 1×10⁷ CFU/ml bacterial suspension was dispensed onto each dish (1×10⁶ CFU per plate), which contained three sterile glass beads. The beads were swirled to spread the inoculum evenly across the surface of the plate. Dry substrate disks were then placed in the center of each plate using sterile forceps. Dry enzyme disks were then placed on top of the dry substrate disks and each plate was wrapped with parafilm and incubated at 26.7° C. Zones of inhibition surrounding each treatment disk were measured after three days. Data were analyzed by analysis of variance in R Studio version 3.3.0 (R Studio, Boston, MA) using packages lme4 and lsmeans, and mean separations were done using Tukey's honest significant difference analysis. Means were considered significantly different at P<0.05.

TABLE 1
Optimization of lactoperoxidase and glucose oxidase in dry enzyme disks

| LP:GOx | LP conc | GOx conc. | Nanoparticle | Enzyme conc. in |
|---|---|---|---|---|
| molar ratio | (μg/ml) | (μg/ml) | conc. (mg/ml) | dry disks (nM) |
| 10:1 | 125 | 25.8 | 0.503 | 94 |
| 5:1 | 125 | 51.6 | 0.589 | 110 |
| 1:1 | 125 | 258 | 1.277 | 238 |
| 1:5 | 25 | 258 | 0.943 | 176 |
| 1:10 | 12.5 | 258 | 0.902 | 168 |

TABLE 2
Dry substrate disk compositions

| Component | 1X concentration | 10X concentration |
|---|---|---|
| Potassium iodide | 0.3 mM | 3 mM |
| Ammonium thiocyanate | 0.5 mM | 5 mM |
| Carboxymethyl cellulose | 0.7% | 0.7% |
| Glucose | 50 mM | 500 mM |

To optimize enzyme concentrations for activity against fungi, four enzyme concentrations (1×, 2×, 5×, and 10×) were analyzed in a 1:1 molar ratio of LP:GOx. LP at 1250 μg/ml and GOx at 2580 μg/ml were mixed in a 1:1 ratio (pH 7.4) and the LP+GOx suspension was combined with nanoparticles at 12.77 mg/ml (pH 3) in a 1:1 enzyme:NP volume ratio. Proportional volumes of immobilized enzymes were then added to enzyme disk components listed in Example 2 (see Table 3) to achieve the following enzyme concentrations: 1×=238 nM; 2×=476 nM; 5×=1190 nM; and 10×=2380 nM. Disks were dried under vacuum prior to use.

The dry enzyme disks were analyzed for efficacy against R. solani and F. graminearum. 10× substrate disks (Table 2) were placed on the center of 85-mm plates containing potato dextrose agar. Dry enzyme disks were then placed on top of substrate disks, one per plate, followed by a 7 mm fungal culture plug placed directly on top of the enzyme disk mycelia side down. Fungal colony diameters were measured after five days (R. solani) and six days (F. graminearum).

To optimize activity against oomycetes, three enzyme concentrations (0.0017×, 0.34×, 1× in a 1:1 molar ratio of LP:GOx) were analyzed. Enzymes were immobilized as previously described and proportional volumes of immobilized enzymes were then added to enzyme disk components listed in Example 2 (Table 3) to achieve the following enzyme concentrations: 0.0034×=0.8 nM; 0.68×=161 nM; and 2×=476 nM. Disks were dried prior to use.

The dry enzyme disks were analyzed for efficacy against P. ultimum. 10× substrate disks (see Table 2) were placed on the center of 85-mm plates containing potato dextrose agar. Dry enzyme disks were then placed on top of substrate disks, one per plat, followed by a 7 mm fungal culture plug placed directly on top of the enzyme disk mycelia side down. Oomycete colony diameters were measured after five days.

### Bactericidal Enzyme Ratio Optimization

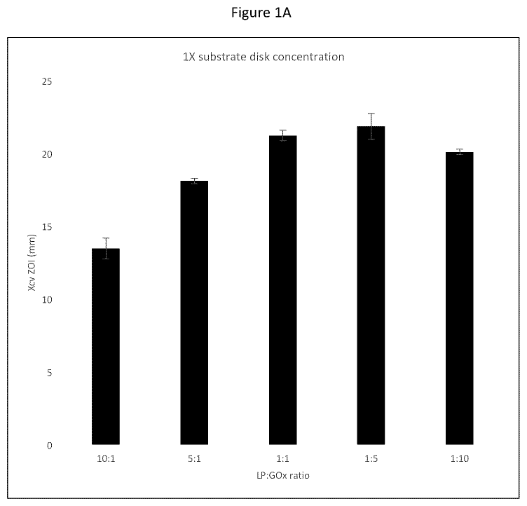

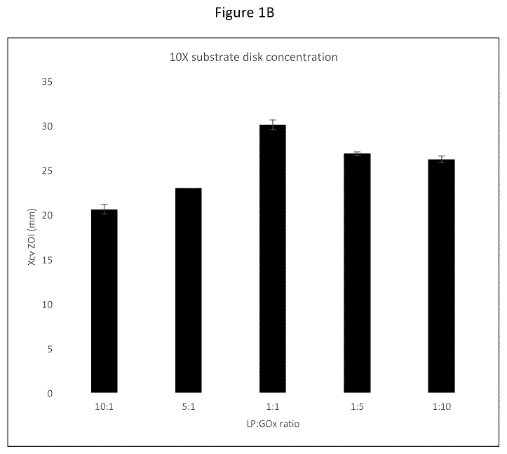

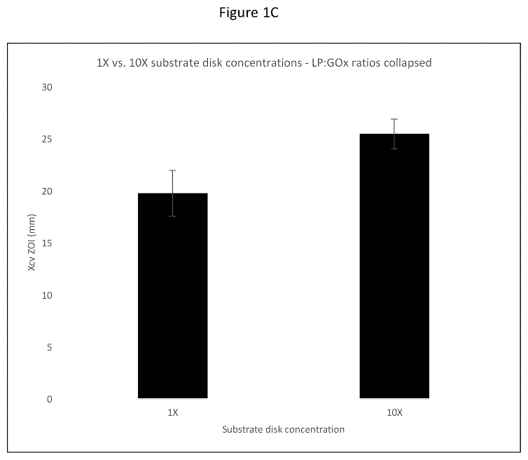

When LP:GOx ratios were analyzed using the 1× substrate concentration, the 1:1 ratio and ratios favoring higher GOx concentrations resulted in the largest zones of inhibition of Xcv (FIG. 1A). This result was not statistically significant based on analysis of variance and Tukey's honestly significant difference (Tukey's HSD, P<0.05) in a first analysis, but was significant in a second analysis with the exception of the 1:10 ratio. A similar pattern was observed when LP:GOx ratios were analyzed using the 10× substrate concentration (see FIG. 1B). In analysis 2, the 1:1 ratio resulted in significantly larger zones of inhibition compared to all other treatments. Ratios of 1:5 and 1:10 also had significantly more efficacy than the 10:1 and 5:1 ratios. The 10× substrate concentration resulted in significantly larger zones of inhibition than the 1× substrate concentration in both analysis (see FIG. 1C). Optimal dry enzyme and substrate disk formulae for maximizing bactericidal efficacy was determined to be a 1:1 LP:GOx molar ratio and 10× substrate concentration.

### Fungicidal Enzyme Concentration Optimization

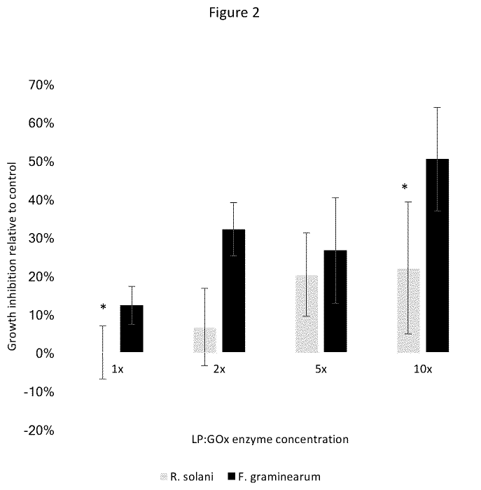

Fusarium graminearum was more sensitive to the dry enzyme disks than Rhizoctonia solani (see FIG. 2). There was a pattern of increasing growth suppression with increasing enzyme concentration observed for both F. graminearum and R. solani. Due to high variance among replicates, few of the observed differences were statistically significant (Tukey's HSD, P<0.05). Rhizoctonia solani growth was inhibited significantly more at the 10× concentration than the 1× concentration (see FIG. 2). Overall, there was a dose effect of LP:GOx enzyme concentration on fungal growth, and the 10× enzyme concentration resulted in the greatest inhibitory effect by reducing growth of R. solani and F. graminearum by 22% and 50%, respectively.

### Optimization of Activity Against Oomycetes

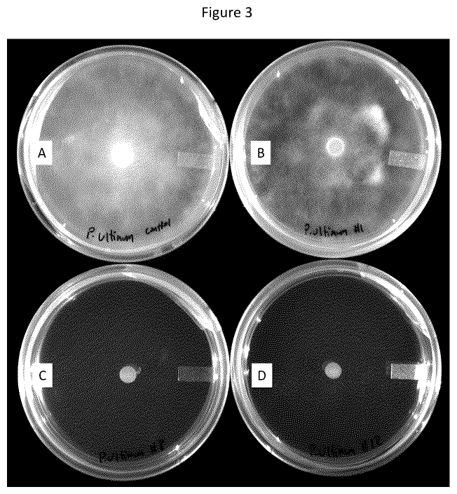

Three LP:GOx concentrations were analyzed in dry enzyme disks for their efficacy against P. ultimum. The lowest concentration (0.0034×) did not inhibit P. ultimum growth relative to the control; however, the 0.68× and 2× concentrations both killed the culture plugs, effectively reducing growth by 100% (see FIG. 3).

### Example 2—Synergy Between Chemical Fungicides and Immobilized LP:GOx

### Materials and Methods

The effect of combining commercial chemical fungicide with immobilized enzymes on microbial growth was analyzed in-vitro on two species of oomycete plant pathogens belonging to the genus Pythium. One isolate each of Pythium ultimum (isolate ‘Geneva 16’) and Pythium aphanidermatum (isolate ‘Pa58’) were both collected in New York State and isolated and identified by Professor Eric Nelson (Cornell University Section of Plant Pathology and Plant-Microbe Biology, Cornell University, Ithaca, NY). https://pppmb.cals.cornell.edu/people/eric-nelson. Isolates were stored on corn meal agar (CMA).

A suspension of lactoperoxidase (LP) and glucose oxidase (GOx) was prepared in a 1:1 M ratio at a concentration of 1.613 μM and pH 7.4. Enzymes were immobilized by combining the 1.613 μM enzyme suspension with a 1.277 mg/ml nanoparticle (NP) suspension (pH 3) in a 1:1 volume ratio. Immobilized enzymes were then combined with solid substrate components as described in Table 3. Separate dry substrate disks were made according to the substrate disk compositions listed in Table 3. Liquid enzyme and substrate disk suspensions were dispensed onto parafilm in 50 μl aliquots and dried under vacuum for approximately 1 hour. Dry enzyme and substrate disks were stored at ambient temperature (approximately 22° C.).

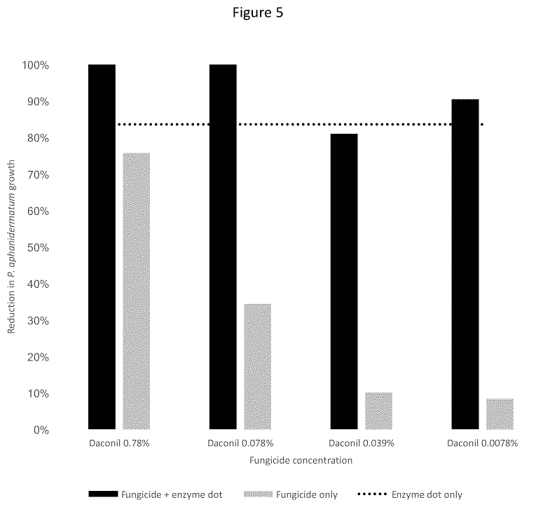

Daconil® fungicide (chlorothalonil 26.6%, manufactured by GardenTech, Palatine, IL, purchased from Amazon, Seattle, WA. cat. no. B000RUGIY0).) Daconil® was mixed with sterile DDI H₂O to create a series of 4 dilutions (0.78% Daconil®, 0.078% Daconil®, 0.039% Daconil®, 0.0078% Daconil®). The highest concentration analyzed (0.78% Daconil®) is twice the highest label rate for vegetables, and was just below the half maximal effective concentration (EC₅₀) value for Daconil® on Pythium ultimum. The EC₅₀ is defined as the concentration of fungicide that induces a response halfway between the baseline and maximum response. The EC₅₀ value for Daconil® on Pythium ultimum was 0.2% based on regression analysis of the data shown in FIG. 5. Based on the regression it was estimated that the full fungicidal concentration is approximately 5% Daconil®.

Fungicides were applied to the center of an 85 mm petri dish containing CMA by stacking two sterile 7 mm filter paper discs on the center of each plate and applying 50 μl of fungicide solution or sterile DDI H₂O (control) to the stacked discs. Treatments that included Daconil®+immobilized enzyme disks were prepared by placing, in a stack on the center of each CMA plate, one substrate disk, one enzyme disk, and 2 filter paper discs with 50 μl fungicide applied as the last step. A 7 mm plug of actively growing Pythium mycelia was placed, mycelia side down, directly on top of the treatment at the center of each plate. The entire analysis was done once for Pythium ultimum and Pythium aphanidermatum.

Treatments were as follows:
Daconil® (0.78%) only
Daconil® (0.078%) only
Daconil® (0.039%) only
Daconil® (0.0078%) only
Control—H₂O on filter paper
Control—1 dry enzyme disk and 1 dry substrate disk+H₂O on filter paper
Daconil® (0.78%)+1 dry enzyme disk and 1 dry substrate disk
Daconil® (0.078%)+1 dry enzyme disk and 1 dry substrate disk
Daconil® (0.039%)+1 dry enzyme disk and 1 dry substrate disk
Daconil® (0.0078%)+1 dry enzyme disk and 1 dry substrate disk

Plates were stored on the bench and colonies allowed to grow for 2 days before two perpendicular colony diameter measurements per plate were recorded. The two perpendicular measurements were averaged for each plate.

TABLE 3
Solid immobilized enzyme and substrate disk compositions

| Enzyme disk composition | | Substrate disk composition | |
|---|---|---|---|
| Component | Concentration | Component | Concentration |
| Potassium iodide | 0.3 mM | Potassium | 0.3 mM |
| | | iodide | |
| Ammonium | 0.5 mM | Ammonium | 0.4 mM |
| thiocyanate | | thiocyanate | |
| Carboxymethyl | 0.7% | Carboxymethyl | 0.7% |
| cellulose | | cellulose | |
| LP + GOx (1:1 ratio) | 81 nM | Avicel | 2.0% |
| | | Glucose | 50M |

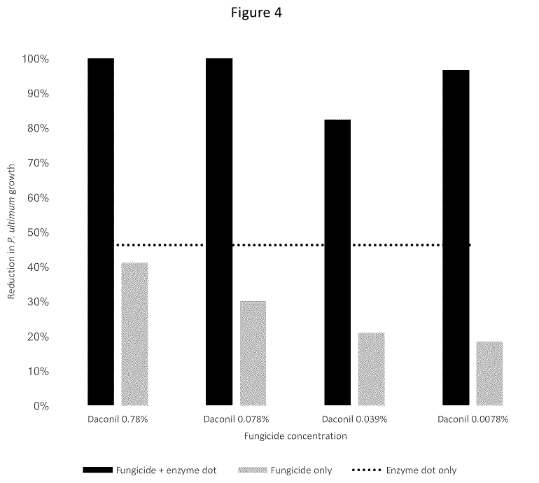

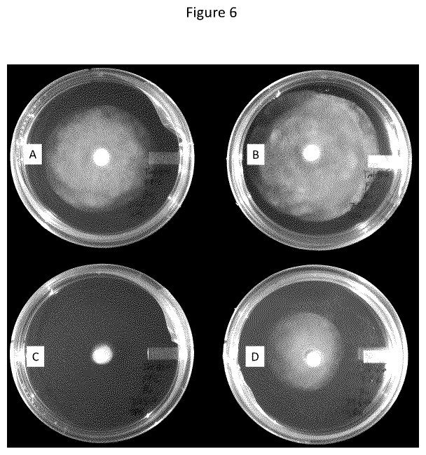

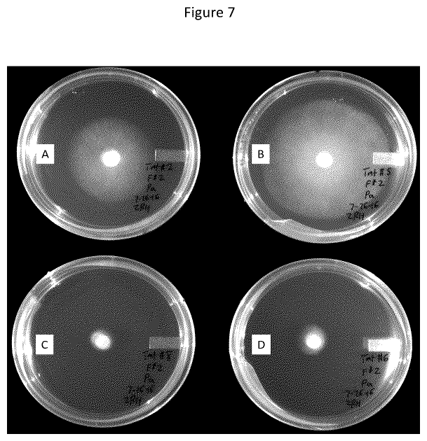

Daconil®+enzyme disk treatments resulted in increased suppression of P. ultimum and P. aphanidermatum compared to Daconil®-only and enzyme disk-only treatments (see FIGS. 4, 5, 6, and 7). The additive effect of Daconil®+enzyme disk was calculated by adding together the reduction in growth due to Daconil® only and the reduction in growth due to enzyme disk only. The enzyme disk-only treatment resulted in a 46% and 84% reduction in P. ultimum and P. aphanidermatum growth, respectively. The additive effect of Daconil®+enzyme disk exceeded 100% for P. aphanidermatum due to its high sensitivity to the enzyme disk. Therefore, the additive and synergistic effects are only reported for P. ultimum (see FIG. 4). Synergistic activity of Daconil®+enzyme disk on P. ultimum ranged from a 13% increase in growth suppression at the highest Daconil® concentration to 32% growth suppression at the lowest Daconil® concentration. Synergism between Daconil® and enzyme disk was observed in each of the four Daconil® concentrations analyzed on P. ultimum (see FIG. 4). Values reported in FIGS. 4 and 5 are the percent reduction in Pythium growth relative to the untreated control.

In-vitro exposure of P. ultimum and P. aphanidermatum to the commercial chemical fungicide Daconil® at four concentrations, enzyme disk only, and Daconil®+enzyme disk resulted in reduced mycelial growth in every treatment relative to the untreated control. The effect of the solid substrate disk only and sterile filter paper+H₂O were analyzed as controls and neither were found to inhibit mycelial growth. The synergistic effect of the combination of Daconil® and enzyme disk on inhibition of P. ultimum was observed at all four Daconil® concentrations used (see FIG. 4). This showed that Daconil®, or chlorothalonil-containing products, had enhanced efficacy through the inclusion of immobilized enzyme disk. Additionally, less chemical fungicide may be used in the presence of enzyme disks to achieve the same level of fungal suppression or disease control as higher fungicide application rates in the absence of enzyme disks.

Synergism between Daconil® and enzyme disks could not be calculated for P. aphanidermatum due to the high sensitivity of this species to the enzyme disk used in this analysis. However, treatments of Daconil®+enzyme disk consistently resulted in greater suppression of P. aphanidermatum growth compared to treatments of Daconil® only and enzyme disk only (see FIG. 5). The effect of lower enzyme concentrations of LP:GOx in immobilized enzyme disks with and without Daconil® on P. aphanidermatum may also be analyzed.

### Example 3—Synergy Between Antibiotics and Immobilized LP:GOx

### Materials and Methods

Antibiotic suspensions (ampicillin) were diluted to four concentrations so that the final amounts applied to sterile filter paper disks were 0.1 μg, 1 μg, 10 μg and 100 μg. The minimum inhibitory concentration (MIC) of ampicillin for Xanthomonas campestris was estimated to be between 10 μg and 50 μg. The MIC is defined as the lowest concentration of antibiotic that completely inhibits growth of the bacteria being evaluated and the minimum bactericidal concentration (MBC) is defined as the lowest concentration of antibiotic at which bacteria are killed. This was not determined for Xanthomonas campestris because resistant colonies persisted at the highest antibiotic concentration. Dry enzyme disks were made according to the formula in Table 3 with the exception of the enzyme concentration which is 238 nM. 10× dry substrate disks were made according to the formula in Table 2. The following treatments were analyzed:
0.1 μg antibiotic only
1 μg antibiotic only
10 μg antibiotic only
100 μg antibiotic only
Control—sterile DDI H₂O on filter paper
Control—1 dry enzyme disk and 1 dry substrate disk+sterile DDI H₂O on filter paper
Control—1 dry substrate disk+sterile DDI H₂O on filter paper
0.1 μg antibiotic+1 dry enzyme disk and 1 dry substrate disk
1 μg antibiotic only+1 dry enzyme disk and 1 dry substrate disk
10 μg antibiotic only+1 dry enzyme disk and 1 dry substrate disk
100 μg antibiotic only+1 dry enzyme disk and 1 dry substrate disk

The analysis was performed by creating bacterial lawns of Xcv on 85-mm petri dishes as described in Example 1. It followed the methods described in Example 2 with the exception of using antibiotic suspensions at four concentrations instead of fungicides. It also measured zones of inhibition of bacterial growth after two days rather than placing 7-mm fungal plugs on top of the treatments and measuring colony diameters.

### Results

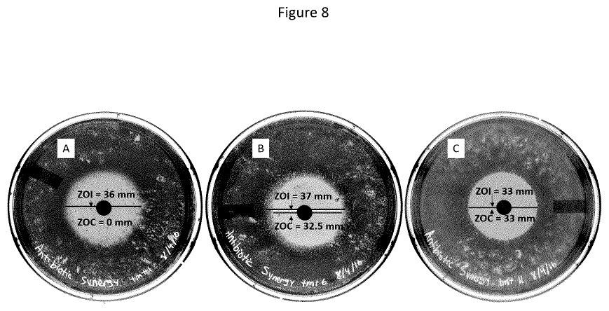

Of the four ampicillin-alone treatments, only the 100 μg ampicillin-alone treatment inhibited the growth of Xcv. This treatment resulted in a ZOI of 36 mm (FIG. 8). 100 μg ampicillin-alone did not result in a ZOC and ampicillin-resistant bacterial colonies could be seen throughout the 36 mm ZOI (FIG. 8). All four of the ampicillin+dry enzyme disk treatments resulted in ZOC ranging from 31 mm to 33.5 mm (Table 4). The 100 μg ampicillin+dry enzyme disk treatment resulted in a ZOC of 32.5 mm with an additional ZOI extending beyond the boundary of the ZOC by an additional 4.5 mm. The 100 μg ampicillin+dry enzyme disk treatment resulted in a greater inhibitory effect against Xcv than either ampicillin alone or enzyme disk alone by clearing all viable bacteria in the ZOC surrounding the treatment and by inhibiting bacterial growth beyond the ZOC. The controls without enzyme did not inhibit growth of Xcv.

TABLE 4
Zones of clearance (ZOC) and zones of inhibition (ZOI) of Xcv
surrounding ampicillin-alone and ampicillin + dry enzyme disk treatments.

| | ZOC | ZOI |
|---|---|---|
| Treatment | (mm) | (mm) |
| 100 ug ampicillin | 0 | 36 |
| 10 ug ampicillin | 0 | 0 |
| 1 ug ampicillin | 0 | 0 |
| 0.1 ug ampicillin | 0 | 0 |
| Control - fp + H2O | 0 | 0 |
| 100 ug ampicillin + enzyme | 32.5 | 37 |
| disk | | |
| 10 ug ampicillin + enzyme disk | 33.5 | 0 |
| 1 ug ampicillin + enzyme disk | 31 | 0 |
| 0.1 ug ampicillin + enzyme | 32.5 | 0 |
| disk | | |
| Control - substrate disk + fp + | 0 | 0 |
| H2O | | |
| Control - enzyme disk | 33 | 0 |

### Example 4—Plant Pathogen Control Using Stabilized Biocidal Enzymes and Biocidal Vegetable Extracts

Essential oils, such as tea tree oil, have antimicrobial properties. Tea tree oil (TTO) in combination with stabilized biocidal enzymes, controlled plant pathogens as shown using the plant pathogenic oomycete Pythium ultimum. Dilutions of TTO were impregnated into filter paper (FP) disks, combined with stabilized biocidal enzyme disks, and placed onto cornmeal agar plates with a plug of P. ultimum culture. The reduction in colony growth was measured and compared to colony growth in the absence of biocidal enzymes and TTO.

### Materials and Methods

Stabilized biocidal enzyme disk preparation. Dry enzyme disks were made by combining 3 μl KI (IM), 5 μl NH₄SCN (IM), 175 μl 4% carboxymethyl cellulose (CMC), 295 μl stabilized lactoperoxidase+glucose oxidase (LPO+GOx) (119 nM+152.2 nM), and 3 μl blue food dye brought up to a final volume of 1 ml with DDI H₂O. Enzyme stabilization was performed as follows: lactoperoxidase (LPO) (125 μg/ml, pH 7.4) and glucose oxidase (GOx) (330 μg/ml, pH 7.4) were mixed to achieve a 1:1.3 M LPO:GOx ratio and stored on ice. Magnetite nanoparticles (NP) (1.277 mg/ml, pH 3, approximately 5 ml stock) were ultrasonicated at 40% amplitude for 1 min, cooled to ambient temperature (approximately 21° C.) in a water bath, and pipette mixed with the LPO:GOx enzyme suspension in a 1:1 enzyme:NP ratio. Dry substrate disks were made my combining 30 μl KI (IM), 50 μl NH₄SCN (1M), 350 μl 4% CMC, 500 μl glucose (IM), and 3 μl red food dye brought up to a final volume of 1 ml with DDI H₂O. Dyes were included to differentiate enzyme disks from the substrate disks and were not biologically active or structural components of the disks. Each solution was pipette mixed several times and vortexed briefly. Solutions were dispensed in 50 μl aliquots onto parafilm and dried at ambient temperature in a vacuum oven containing desiccant at −50 kPa. After approximately 2 hours, dry enzyme and substrate disks were stored in the dark at 4° C. until use.

Tea tree oil disk preparation and culture growth assay. Tea tree oil (Active Ingredient (AI): tea tree oil 100%, Mason Natural, Miami Lakes, FL) (TTO) was diluted in sterile DDI H₂O. Whatman® qualitative grade 1 filter paper (Sigma-Aldrich) was cut into 7 mm disks using a 3-hole punch, and disks were autoclaved prior to use. FP disks were impregnated with 5 μl of each of four TTO dilutions. Control FP disks were impregnated with 5 μl sterile DDI H₂O. TTO and enzyme disk interactions were tested by placing one substrate disk on the center of a petri dish containing corn meal agar, followed by one enzyme disk, one TTO-impregnated FP disk, and one culture plug mycelia-side down. Final enzyme concentrations in enzyme disks were 4 nM for P. ultimum. Culture plugs of P. ultimum measured 7 mm in diameter. Each experiment included the same TTO dilution series plated without substrate and enzyme disks, as well as a substrate+enzyme disk-only treatment and a non-treated control. All plates, including controls, contained a FP disk, and each treatment was replicated once. Plates were left on the bench at ambient temperature for 2 days. At that time, control colonies had nearly grown to the plate edge. Two perpendicular colony diameter measurements were recorded for P. ultimum.

### Results

Tea tree oil combined with the stabilized enzyme formulation resulted in a statistically synergistic effect at the two lowest TTO concentrations and was additive at the two highest concentrations (Table 5). The combined effects were significantly greater than the effects of the enzyme formulation alone and three of the four TTO concentrations alone. The highest TTO concentration alone produced a significantly greater effect than enzyme formulation alone, and was not significantly different from any of the four combined effects (Table 5).

TABLE 5
Inhibition of Pythium ultimum by the stabilized LPO formulation alone and
in combination with tea tree oil using TTO-impregnated filter paper disks.

| | | | | | | Combined | |
|---|---|---|---|---|---|---|---|
| Tea | Reduction in growth^{a} | | | | | effect | |
| tree oil | (+) | | (−) | | Tukey's | (observed − | Combination |
| dose | enzyme | SD | enzyme | SD | HSD | expected)^{b} | result |
| 30% | 100% a^{c} | 0.0% | 91% a | 12.1% | 23.4% | 0% | additive |
| 20% | 100% a | 0.0% | 66% b | 9.4% | | +7% | additive |
| 15% | 100% a | 0.0% | 45% bc | 2.1% | | +28% | synergistic |
| 10% | 94% a | 6.6% | 31% c | 1.6% | | +36% | synergistic |
| 0% | 27% c | 4.7% | NA | NA | | | |

^{a}Reduction in growth relative to non-treated controls. Each value is the mean of two replicates.
^{b}Difference between observed effect of fungicide (+) enzyme formulation and expected additive effect. Expected value calculated by adding the effect of fungicide alone and the effect of the enzyme formulation alone for each fungicide concentration.
^{c}Means followed by the same letter are not significantly different, Tukey's HSD (P < 0.0

### Example 5—Pathogenic Nematode Control Using Biocidal Stabilized Enzymes and Stabilized Emulsified Tea Tree Oil

Root-knot nematodes from the genus Meloidogyne infect a wide array of plants, including woody crops and vegetables, causing yield loss. The cyst nematode Heterodera schachtii causes growth retardation in infected plants and can cause massive yield loss at high population densities. Control of nematode populations is vital to reducing losses associated with reduced crop yields. Stabilized biocidal enzymes combined with TTO control plant pathogens but TTO is not miscible in water and difficult to combine with biocidal enzymes. In this example, microencapsulated TTO was combined with stabilized biocidal enzymes for the control of plant pathogens. Nematode juveniles, eggs, and cysts were incubated with treatments containing biocidal stabilized enzymes, TTO, or both, to measure plant pathogenic nematodes killing or control.

### Materials and Methods

Preparation of microencapsulated TTO and stabilized biocidal enzymes. Stabilized microencapsulated TTO (40%, 40× strength) was prepared by combining 2 ml of a 2% (w/w) solution of EHM (Ethyl hydroxyethyl cellulose EHM300, Akzonobel), 4 ml TTO (pure), and 4 ml DDI H₂O. This solution was sonicated two times for 1 minute each at 40% amplitude, ¼th inch horn. The 20×TTO substrate solution was made by combining 100 μl stabilized EHM TTO mix (40% TTO), 500 μl glucose (IM), 3 μl red food dye, and brought up to a final volume of 1 ml with DDI H₂O. The standard substrate solution was made by combining 500 μl glucose (1M), 3 μl red food coloring dye, and brought up to a final volume of 1 ml with DDI H₂O. The enzyme solution was made by combining 3 μl KI (IM), 5 μl NH₄SCN (1M), 70 μl 4% carboxymethyl cellulose (CMC), 295 μl stabilized lactoperoxidase+glucose oxidase (LPO+GOx) (119 nM+152.2 nM), and 3 μl blue food dye brought up to a final volume of 1 ml with DDI H₂O. Enzyme stabilization was performed as follows: LPO (125 μg/ml, pH 7.4) and GOx (330 μg/ml, pH 7.4) were mixed to achieve a 1:1.3 M LPO:GOx ratio and stored on ice. Magnetite nanoparticles (NP) (1.277 mg/ml, pH 3, approximately 5 ml stock) were ultrasonicated at 40% amplitude for 1 min, cooled to ambient temperature (approximately 21° C.) in a water bath, and pipette mixed with the LPO:GOx enzyme suspension in a 1:1 enzyme:NP ratio. Each solution was pipette mixed several times and vortexed briefly. Solutions were stored in the dark at 4° C. until use.

Control of plant pathogenic nematodes using microencapsulated TTO and stabilized biocidal enzymes. For the nematode juvenile analyses, a stock suspension of Meloidogyne incognita juveniles was prepared at a concentration of 300 juveniles/ml. Four treatment solutions were prepared as follows (1 ml total volume): Treatment solution (Tmt) 1=33 μl enzyme solution (1.96 nM lactoperoxidase+2.51 nM glucose oxidase)+33 μl standard substrate solution (500 mM glucose)+934 μl water, Tmt 2=33 μl 0.066% tea tree oil (TTO) solution+967 μl water, Tmt 3=33 μl enzyme solution (1.96 nM lactoperoxidase+2.51 nM glucose oxidase)+33 μl 0.066% TTO solution+934 μl water, and Tmt 4 (control)=1000 μl water. Each treatment solution was combined with 1 ml of the M. incognita stock suspension in a 10 ml beaker for 300 juveniles per experiment. Analyses were triplicated for each treatment resulting in 12 samples in total. They were stored individually in tightly sealed boxes to reduce volume loss due to evaporation at room temperature for three days. After three days, 100 juveniles were removed from each sample and counted as live or dead. If movement was observed during the count, the juvenile was counted as alive. If no movement was observed, the juvenile was counted as dead.

In the nematode cyst analyses, each sample (10 ml beaker) received approximately 50 Heterodera schachtii cysts. Cysts were hand-picked under a dissecting microscope and added to each beaker. Four treatment solutions were prepared as follows (2 ml total volume): Tmt1=33 μl enzyme solution (1.96 nM lactoperoxidase+2.51 nM glucose oxidase)+33 μl standard substrate solution (500 mM glucose)+1934 μl water, Tmt2=33 μl 0.066% tea tree oil (TTO) solution+1967 μl water, Tmt3=33 μl enzyme solution (1.96 nM lactoperoxidase+2.51 nM glucose oxidase)+33 μl 0.066% TTO solution+1934 μl water, and Tmt4 (control)=2000 μl water. Each sample with 50 cysts received 2 ml of treatment solution (2 ml total experimental volume). Analyses were triplicated for each treatment resulting in 12 samples in total. Samples were stored individually in boxes at room temperature for 1 week. After 1 week, 300 μl of the sample volume was removed and the number of live or dead juveniles was counted as previously described.

In the nematode egg analyses, a stock suspension of H. schachtii eggs was prepared at a concentration of 1200 eggs/ml. Each sample (10 ml beaker) received 800 μl of the egg suspension for 960 eggs per experimental unit. Four treatment solutions were prepared as follows (1.2 ml total volume): Tmt1=33 μl enzyme solution (1.96 nM lactoperoxidase+2.51 nM glucose oxidase)+33 μl standard substrate solution (500 mM glucose)+1134 μl water, Tmt2=33 μl 0.066% tea tree oil (TTO) solution+1167 μl water, Tmt3=33 μl enzyme solution (1.96 nM lactoperoxidase+2.51 nM glucose oxidase)+33 μl 0.066% TTO solution+1134 μl water, and Tmt4 (control)=1200 μl water. Each sample with 800 μl H. schachtii egg suspension received 1.2 ml of treatment solution (2 ml total experimental volume). Analyses were triplicated for each treatment resulting in 12 samples in total. Samples were stored individually in boxes at room temperature for 1 week. After 1 week, 300 μl of the sample volume was removed and the number of live or dead juveniles was counted as previously described.

### Results

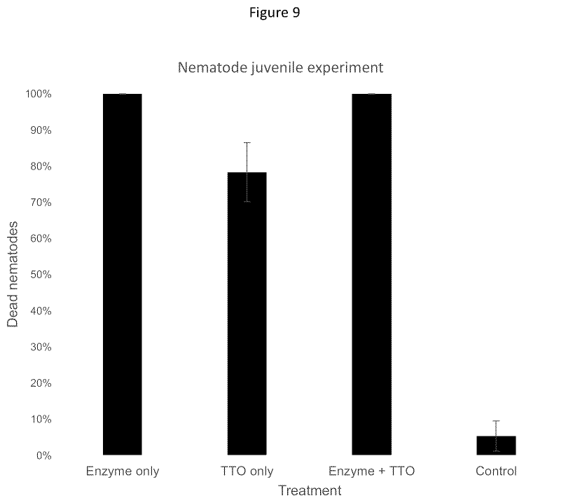

Results from each of the analyses showed effective control of nematodes by stabilized enzymes. FIG. 9 shows the result of the nematode juvenile experiment for which the treatments with stabilized enzymes alone and stabilized enzymes with tea tree oil showed 100% effectiveness at killing M. incognita juveniles. 78% of the nematode juveniles were killed in a solution of tea tree oil alone. Only 5% of nematode juveniles were killed in the control sample.

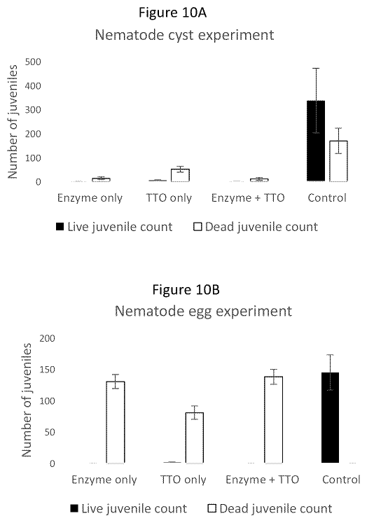

FIGS. 10A and 10B show the results of the nematode egg and cyst analyses. In FIG. 10A, the total number of nematode juveniles hatched from H. schachtii cysts, both live and dead, observed in the treatments with stabilized enzymes, TTO, or both is greatly reduced from the total number of nematodes observed in the control solution. The treatments inhibit hatching from the cysts. The enzyme treatment is more inhibitory to hatching than TTO alone. Juveniles that do hatch from cysts following treatment are nearly all dead by the end of the incubation period. FIG. 10B shows the number of nematode juveniles hatched from eggs, both live and dead, observed in the treatments with stabilized enzymes, TTO, or both. In this case, the treatments do not inhibit hatching, though the juveniles that do hatch are subsequently killed by the treatments during the incubation period.

### Example 6—Plant Pathogen Control Using Stabilized Biocidal Enzymes and Mancozeb

Mancozeb is a non-systemic fungicide used for agriculture to control fungal diseases in a wide array of field crops. An enhanced fungicidal effect from the combination of mancozeb and stabilized biocidal enzymes was shown. Solid culture growth media was amended with mancozeb at four concentrations. Stabilized biocidal enzyme disks and standard substrate disks were placed onto these plates with a plug of P. ultimum or F. graminearum culture. The reduction in colony growth was measured and compared to colony growth in the absence of biocidal enzymes and mancozeb.

### Materials and Methods

Stabilized biocidal enzyme disk preparation. Dry enzyme disks were made by combining 3 μl KI (IM), 5 μl NH₄SCN (IM), 175 μl 4% carboxymethyl cellulose (CMC), 295 μl stabilized lactoperoxidase+glucose oxidase (LPO+GOx) (119 nM+152.2 nM), and 3 μl blue food dye brought up to a final volume of 1 ml with DDI H₂O. Enzyme stabilization was performed as follows: lactoperoxidase (LPO) (125 μg/ml, pH 7.4) and glucose oxidase (GOx) (330 μg/ml, pH 7.4) were mixed to achieve a 1:1.3 M LPO:GOx ratio and stored on ice. Magnetite nanoparticles (NP) (1.277 mg/ml, pH 3, approximately 5 ml stock) were ultrasonicated at 40% amplitude for 1 min, cooled to ambient temperature (approximately 21° C.) in a water bath, and pipette mixed with the LPO:GOx enzyme suspension in a 1:1 enzyme:NP ratio. Dry substrate disks were made my combining 30 μl KI (IM), 50 μl NH₄SCN (1M), 350 μl 4% CMC, 500 μl glucose (1M), and 3 μl red food dye brought up to a final volume of 1 ml with DDI H₂O. Dyes were included to differentiate enzyme disks from the substrate disks and were not biologically active or structural components of the disks. Each solution was pipette mixed several times and vortexed briefly. Solutions were dispensed in 50 μl aliquots onto parafilm and dried at ambient temperature in a vacuum oven containing desiccant at −50 kPa. After approximately 2 hours, dry enzyme and substrate disks were stored in the dark at 4° C. until use.

Mancozeb amended plates and culture growth assays. Corn meal agar (CMA) and potato dextrose agar (PDA) (for P. ultimum and F. graminearum, respectively) were amended with mancozeb flowable with zinc (Active ingredient (AI): mancozeb 37%, Bonide, Oriskany, NY) to achieve final concentrations of 10 mg/1, 5 mg/l, 2 mg/l, 0.5 mg/l, and 0 mg/l (controls). Mancozeb and enzyme disk interactions were analyzed by placing one substrate disk on the center of each petri dish containing PDA amended with mancozeb (F. graminearum) or CMA amended with mancozeb (P. ultimum). These were followed by one enzyme disk and one culture plug mycelia-side down. Final enzyme concentrations in enzyme disks were 4 nM for P. ultimum and 119 nM for F. graminearum and based on results from preliminary enzyme formula optimization experiments. Plugs of P. ultimum measured 7 mm in diameter and plugs of F. graminearum measured 4 mm in diameter.

Preliminary testing revealed that P. ultimum is more sensitive to the enzyme treatment than F. graminearum. Thus, enzyme concentrations and plug sizes were chosen to achieve a measurable growth reduction, without being completely inhibitive, in enzyme-only treatments compared to non-treated controls. Each analysis included the same fungicide dilution series plated without substrate and enzyme disks as well as a substrate+enzyme disk-only treatment and a non-treated control. Each treatment was replicated once. Plates were left on the bench at ambient temperature for 2 days for P. ultimum, and 5 days for F. graminearum. Following the incubation period, control colonies had nearly grown to the plate edge. Two perpendicular colony diameter measurements were recorded for P. ultimum. Colonies of F. graminearum measured using the public domain image processing program ImageJ.

### Results

Mancozeb combined with the stabilized enzyme formulation resulted in a statistically synergistic effect on P. ultimum at the lowest fungicide concentration and was additive at the three highest concentrations (Table 6). The combined effects were significantly greater than the effects of the enzyme formulation alone and all four fungicide concentrations alone. The effect of the enzyme formulation alone was significantly greater than the three lowest fungicide concentrations alone, but was not significantly different from the highest concentration alone (Table 6). Combined activity against F. graminearum was additive at all four fungicide concentrations (Table 6). The combined effects were significantly greater than the corresponding fungicide concentrations alone at the three highest concentrations. The effect of the enzyme formulation alone was not significantly different from any of the combined effects nor the fungicides alone (Table 6).

TABLE 6
Inhibition of Pythium ultimum and Fusarium graminearum by the stabilized LPO
formulation alone and in combination with mancozeb using fungicide-amended media.

| | | | | | | Combined | |
|---|---|---|---|---|---|---|---|
| Mancozeb | Reduction in P. ultimum growth^{a} | | | | | effect | |
| concentration | (+) | | (−) | | Tukey's | (observed − | Combination |
| (mg/l) | enzyme | SD | enzyme | SD | HSD | expected)^{b} | result |
| 10 | 53% a^{c} | 0.3% | 23% d | 1.7% | 6.04% | +4% | additive |
| 5 | 45% b | 0.5% | 16% e | 0.4% | | +3% | additive |
| 2 | 34% c | 1.3% | 10% e | 0.8% | | −2% | additive |
| 0.5 | 35% c | 3.7% | 2% f | 0.5% | | +7% | synergistic |
| 0 | 26% d | 0.9% | NA | NA | | | |

| | | | | | | Combined | |
|---|---|---|---|---|---|---|---|
| Mancozeb | Reduction in F. graminearum growth^{a} | | | | | effect | |
| concentration | (+) | | (−) | | Tukey's | (observed − | Combination |
| (mg/l) | enzyme | SD | enzyme | SD | HSD | expected)^{b} | result |
| 10 | 58% a^{c} | 5.7% | 26% bcd | 6.0% | 32.4% | +3% | additive |
| 5 | 53% ab | 15.6% | 17% cd | 5.4% | | +7% | additive |
| 2 | 51% ab | 5.3% | 1% d | 5.1% | | +21% | additive |
| 0.5 | 39% abc | 10.9% | 6% cd | 3.8% | | +4% | additive |
| 0 | 29% abcd | 8.9% | NA | NA | | | |

^{a}Reduction in growth relative to non-treated controls. Each value is the mean of two replicates.
^{b}Difference between observed effect of fungicide (+) enzyme formulation and expected additive effect. Expected value calculated by adding the effect of fungicide alone and the effect of the enzyme formulation alone for each fungicide concentration.
^{c}Means followed by the same letter are not significantly different, Tukey's HSD (P < 0.05).

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1.-73. (canceled)

74. A liquid biocidal composition, comprising;
a. a polymeric solvatable matrix formulated with self-assembled mesoporous aggregates of magnetic nanoparticles comprising a stable hydrogen peroxide producing enzyme and a stable free radical producing enzyme;
b. a component having a first substrate for said hydrogen peroxide producing enzyme and a second substrate for said free radical producing enzyme; and
c. a chemical biocide;
wherein said composition is essentially inactive, wherein mixing said polymeric solvatable matrix and said component activates said composition and results in said substrate for said hydrogen peroxide producing enzyme being oxidized into hydrogen peroxide, wherein said hydrogen peroxide acts as a substrate for said free radical producing enzyme, and wherein said free radicals are produced having biocidal activities.

75. The liquid biocidal composition of claim 74, wherein said composition is fungicidal, wherein said chemical biocide is a chemical fungicide, wherein the final chemical fungicide concentration is between about 10% and 2500% of the half maximal effective concentration (EC₅₀).

76. The liquid biocidal composition of claim 75, further comprising a chemical antibiotic.

77. The liquid biocidal composition of claim 74, wherein said free-radical-producing enzyme and hydrogen peroxide producing enzyme are contained in said mesoporous aggregates of magnetic nanoparticles in up to about 100% of saturation capacity.

78. The liquid biocidal composition of claim 74, wherein said hydrogen peroxide generating enzyme is an oxidase.

79. The liquid biocidal composition of claim 74, wherein said oxidase is glucose oxidase or alcohol oxidase.

80. The liquid biocidal composition of claim 75, wherein said chemical fungicide is selected from the group consisting of mefenoxam, myclobutanil, chlorothalonil, prothioconazole, trifloxystrobin, propiconazole, mancozeb, and copper.

81. The liquid biocidal composition of claim 75, wherein said chemical fungicide is chlorothalonil.

82. The liquid biocidal composition of claim 75, wherein said chemical fungicide is mancozeb.

83. The liquid biocidal composition of claim 74, wherein said composition is bactericidal, wherein said chemical biocide is a chemical antibiotic, wherein said chemical antibiotic has a final concentration between about 1 and 100% of the minimum inhibitory concentration (MIC) or minimum bactericidal concentration (MBC).

84. The liquid biocidal composition of claim 83, wherein said chemical antibiotic is selected from the group consisting of ampicillin, streptomycin, vancomycin, and copper.

85. The liquid biocidal composition of claim 74, wherein said composition is nematocidal and said chemical biocide as a nematocide.

86. The liquid biocidal composition of claim 85, wherein said composition is effective against a nematode that is selected from the group consisting of Meloidogyne species (spp.), Heterodera spp., Globodera spp., Pratylenchus spp., Helicotylenchus spp., Radopholus similis, Ditylenchus dipsaci, Rotylenchulus reniformis, Xiphinema spp, Aphelenchoides spp., Toxocara spp., Bursaphelenchus xylophilus, and Trichinella spiralis.

87. The liquid biocidal composition of claim 85, wherein said composition is effective against a Meloidogyne spp.

88. The liquid biocidal composition of claim 85, wherein said composition is effective against Trichinella spiralis.

89. The liquid biocidal composition of claim 85, wherein said composition is effective against a Toxocara spp.

90. An agricultural product, comprising the liquid biocidal composition of claim 74.

91. A pesticide product, comprising the liquid biocidal composition of claim 74.

92. A method of improving a plant product yield with the liquid biocidal composition of claim 74, comprising mixing said polymeric solvatable matrix with said component and said chemical biocide and applying said liquid biocidal composition to said plant prior to or during the planting or germination of said plant.

93. A method of producing the liquid biocidal composition of claim 74, comprising the step of formulating said polymeric solvatable matrix with a material selected from the group consisting of water-solvatable cellulose derivatives, alginate derivatives, and chitosan derivatives.

94. A method of protecting an agricultural product from a pathogen with the liquid biocidal composition of claim 74, comprising mixing said polymeric solvatable matrix with said component and said chemical biocide and applying said liquid biocidal composition to said agricultural product.

95. The method of claim 94, wherein said pathogen is a plant, animal, or human pathogen.

96. The method of claim 94, wherein said pathogen is a bacterium, fungus, oomycete, or a nematode.

97. The method of claim 96, wherein said fungus is a Fusarium species.

98. The method of claim 96, wherein said oomycete is selected from the group consisting of Pythium species and Phytophthora species.

99. The method of claim 96, wherein said bacterium is Xanthomonas campestris, Clavibacter michiganensis, Acidovorax avenae, Pseudomonas viridiflava, Pseudomonas syringae, Escherichia coli, Salmonella species, or Listeria species.

100. A method of reducing or eliminating damping off in a plant with the liquid biocidal composition of claim 74, comprising mixing said polymeric solvatable matrix with said component and said chemical biocide and applying to said plant prior to or during the planting or germination of said plant.
